(19) United States
(12) Patent Application Publication
(10) Pub. No.: US 2022/0005547 A1
(43) Pub. Date: Jan. 6, 2022

(54) MULTIPLEXED DROPLET-BASED SEQUENCING USING NATURAL GENETIC BARCODES

(71) Applicant: The Regents of the University of California, Oakland, CA (US)

(72) Inventors: Chun Ye, San Francisco, CA (US); Meena SUBRAMANIAM, Fremont, CA (US); Rachel GATE, San Francisco, CA (US); Sasha TARG, San Francisco, CA (US)

(73) Assignee: The Regents of the University of California, Oakland, CA (US)

(21) Appl. No.: 17/294,977

(22) PCT Filed: Dec. 10, 2019
(86) PCT No.: PCT/US2019/065522

§ 371 Date: May 18, 2021

Related U.S. Application Data
(60) Provisional application No. 62/777,590, filed on Dec. 10, 2018.

(51) Int. Cl. G16B 20/20; G16B 5/20; C12Q 1/6809; G16B 25/10; C12Q 1/6874

(52) U.S. Cl. CPC G16B 20/20; G16B 5/20; C12Q 1/6874; G16B 25/10; C12Q 1/6809

(57) ABSTRACT

Techniques for multiplexed droplet-based sequencing using natural genetic barcodes. An exemplary technique determining a unique variant combination associated with each subject from a plurality of subjects, determining a presence of a plurality of target variants in sequence reads grouped into partitions, and performing a sample demultiplexing and multiplet detection technique (demuxlet) on the sequence reads to: (i) remove sequence reads for partitions that are determined to be doublets based on a first statistical comparison of the unique variant combination associated with each subject and the plurality of target variants in the sequence reads, and (ii) assign a subject from the plurality of subjects to the remaining partitions remaining based on a second statistical comparison of the unique variant combination associated with the each subject from the plurality of subjects and the plurality of target variants in the sequence reads.

### PRIORITY CLAIM

This application is the U.S. National Stage Entry of PCT/US2019/065522 filed Dec. 10, 2019, which claims priority from and the benefit of U.S. Provisional Application No. 62/777,590 filed on Dec. 10, 2018, titled “Multiplexed Droplet-Based Sequencing Using Natural Genetic Barcodes” the entire contents of each of which are incorporated herein by reference for all purposes.

### STATEMENT OF GOVERNMENT SUPPORT

The invention was made with government support under Grants No. R01 AR071522 and R21 AI133337 awarded by the National Institutes of Health. The government has certain rights in the invention.

### FIELD OF THE INVENTION

The present disclosure relates generally to molecular barcoding, and more particularly, to techniques for multiplexed droplet-based sequencing using natural genetic barcodes.

### BACKGROUND

Cells are the basic building blocks of all living organisms. Many organisms are comprised of millions of cells that come in different types, sub-types and activity states, to provide structure for the organism, take in nutrients from food, convert those nutrients into energy, and carry out specialized functions. Cells may be classified based on various characteristics including shape, location, function, or molecular content, such as protein, messenger ribonucleic acid (mRNA), genomic DNA, or chromatin state. RNA is particularly informative, as cells express thousands of different RNAs during various states of cell activity. Conventional approaches to classify or profile the molecular content of cells, including cells from complex tissues such as the heart or brain, use expression profiling frameworks where thousands of cells from one sample are processed and analyzed in parallel. Such single cell expression profiling frameworks typically use microfluidic devices to encapsulate each cell or the nucleus of each cell in an individual partition (e.g., an individual micro-reaction chamber) and associate the molecular contents of each cell with a barcode unique to the origin of each cell (i.e., the individual partition containing the cell). Thereafter, sequencing techniques measure the abundance and state of molecular contents of each cell and use the cell barcodes to determine which cell or partition was the origination of the molecular content.

Single cell expression profiling workflows have substantially increased the throughput of cell capture and library preparation enabling the simultaneous classification or profiling (e.g., transcriptomic profiling) of thousands of cells in a single test run. Improvements in biochemistry and microfluidics continue to increase the number of cells and molecular content profiled per test run but standard workflows are limited to processing cells for a single individual.

### BRIEF SUMMARY

The invention recognizes that, for differential expression and population genetics studies, sequencing thousands of cells each from multiple subjects would better capture inter-individual variability than sequencing more cells from a few individuals. The invention provides an approach that allows for molecular content to be distinguished not only between cells but also between subjects, which allows for pooling of cells from multiple different subjects in one microfluidic run, which results in lower per-sample library preparation cost and eliminates confounding effects. Furthermore, with aspects of the invention, partitions containing multiple cells from different subjects can be detected and pooled cells can be loaded at higher concentrations, which enables additional reduction in per-cell library preparation cost. Accordingly, the invention provides new techniques for sample-multiplexed droplet-based sequencing.

The different aspects of the invention allow for parallel profiling of cellular information, such as the expression of nucleic acids or proteins, from a large number of subjects simultaneously. The methods include encapsulating genetically diverse cell samples in separate droplet partitions, where cellular nucleic acids are labeled with partition-specific codes, and performing bulk sequence analysis of the labeled nucleic acids. The sequence reads reveal both the information being profiled, e.g., gene expression, as well as the source droplet in which the labeling occurred. The sequence reads also contain genotypic information that allows identification of the subject whose cellular material was contained in the source droplet. By cross-referencing each subject's unique genotypic information with the identifying code of each partition, the profiled data from each droplet partition can be mapped to an individual subject. Consequently, the methods of the invention permit simple and rapid analysis of 20,000 or more different biological samples while limiting the number of manipulations required and resources consumed.

In that manner, the invention combines the use of predetermined genotypic information that allows identification of individual subjects from which samples are derived and barcoding that allows identification of samples within an assay. Combining these two means of identification allows large numbers of samples to be processed by bulk sequencing to obtain new biological information, such as expression of nucleic acids and proteins. The sequencing reads are then deconvoluted bioinformatically to determine the subject that provided the source material for each sample and ascribe the new biological information to a specific sample from a specific subject.

In embodiments of the invention as described herein, techniques are provided (e.g., a method, a system, non-transitory computer-readable medium storing code or instructions executable by one or more processors) for sample-multiplexed droplet-based sequencing using natural genetic barcodes.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect is directed to a method that includes: obtaining, by a data processing system, genotypes of multiple different loci for a plurality of subjects, where the different loci comprise a predetermined set of target variants that provide a unique variant combination associated with each subject from the plurality of subjects; obtaining, by the data processing system, sequence reads for nucleic acid from target molecules of the plurality of subjects, where the sequence reads comprise nucleic acid sequences with associated partition-specific barcodes that identify a partition from which each sequence read originated; deconvoluting, by the data processing system, the sequence reads by the partition-specific barcodes to group the sequence reads by the partition from which each sequence read originated; determining, by the data processing system, a presence of a plurality of target variants in the sequence reads grouped into each of the partitions; and assigning, by the data processing system, at least one subject from the plurality of subjects to one or more partitions based on a statistical comparison of the unique variant combination associated with each subject from the plurality of subjects and the plurality of target variants in the sequence reads grouped into the one or more partitions. Other embodiments of this aspect include corresponding computer systems, apparatus, and computer programs recorded on one or more computer storage devices, each configured to perform the actions of the methods.

Implementations may include one or more of the following features. The method where the statistical comparison comprises: (i) determining a number of the sequence reads grouped into each of the partitions that overlap with the target variants of the unique variant combination associated with each subject, and (ii) determining a maximum likelihood that the sequence reads grouped into each of the partitions originated from each subject based on the determined number of the sequence reads grouped into each of the partitions that overlap with the target variants of the unique variant combination associated with each subject. The method where the variants from the set of target variants are single nucleotide polymorphisms (SNPs). The method where the deconvoluting comprises organizing the sequence reads by the partition-specific barcodes into a matrix of sequence reads and partitions.

Implementations may include one or more of the following features. The method may also include prior to the assigning: determining, by the data processing system, whether the sequence reads grouped into each of the partitions originate from more than one subject from the plurality of subjects; when the sequence reads grouped into a partition do originate from more than one subject from the plurality of subjects, the sequence reads associated with the partition are removed from the matrix; and when the sequence reads grouped into a partition do not originate from more than one subject from the plurality of subjects, performing the assigning of a subject to the partition based on the statistical comparison of the unique variant combination associated with the each subject from the plurality of subjects and the plurality of target variants in the sequence reads grouped into the partition.

Implementations may include one or more of the following features. The method where determining whether the sequence reads grouped into each of the partitions originate from more than one subject from the plurality of subjects comprises generating a mixture model to calculate a likelihood that the sequence reads grouped into each partition originates from more than one subject from the plurality of subjects. The method further comprises compiling, by the data processing system, a set of sequence data for each subject of the plurality of subjects, where the set of sequence data comprises the sequence reads grouped into each partition assigned to each subject of the plurality of subjects, and the compiling comprises organizing the sequence reads by the partition-specific barcodes and the plurality of target variants into a matrix of sequence reads, partitions, and subjects for further analysis.

One general aspect is directed to a method that includes: obtaining, by a data processing system, genotypes of multiple different loci for a plurality of subjects, where the different loci comprise a predetermined set of target variants that provide a unique variant combination associated with each subject from the plurality of subjects; obtaining, by the data processing system, sequence reads for nucleic acid from the plurality of subjects, where the sequence reads comprise nucleic acid sequences with associated partition-specific barcodes that identify a partition from which each sequence read originated; deconvoluting, by the data processing system, the sequence reads by the partition-specific barcodes to group the sequence reads by the partition from which each sequence read originated where the deconvoluting comprises organizing the sequence reads by the partition-specific barcodes into a matrix of sequence reads and partitions; determining, by the data processing system, a presence of a plurality of target variants in the sequence reads grouped into each of the partitions, where the plurality of target variants determined in the sequence reads grouped into each of the partitions are added to the matrix of sequence reads and partitions; and performing, by the data processing system, a sample demultiplexing and multiplet detection technique (demuxlet) on the sequence reads in the matrix to: (i) remove sequence reads from the matrix for partitions that are determined to be multiplets (e.g., doublets or triplets) based on a first statistical comparison of the unique variant combination associated with each subject and the plurality of target variants in the sequence reads grouped into each of the partitions, and (ii) assign a subject from the plurality of subjects to the partitions remaining in the matrix that have sequence reads based on a second statistical comparison of the unique variant combination associated with the each subject from the plurality of subjects and the plurality of target variants in the sequence reads grouped into the remaining partitions. Other embodiments of this aspect include corresponding computer systems, apparatus, and computer programs recorded on one or more computer storage devices, each configured to perform the actions of the methods.

Implementations may include one or more of the following features. The method where the first statistical comparison comprises generating a mixture model to calculate a likelihood that the sequence reads grouped into each partition originates from more than one subject from the plurality of subjects . The method where the second statistical comparison comprises: (i) determining a number of the sequence reads grouped into each of the partitions that overlap with the target variants of the unique variant combination associated with each subject, and (ii) determining a maximum likelihood that the sequence reads grouped into each of the partitions originated from each subject based on the determined number of the sequence reads grouped into each of the partitions that overlap with the target variants of the unique variant combination associated with each subject.

Implementations may include one or more of the following features. The method where the variants from the set of target variants are single nucleotide polymorphisms (SNPs). The method may also include compiling, by the data processing system, a set of sequence data for each subject of the plurality of subjects, where the set of sequence data comprises the sequence reads grouped into each partition assigned to each subject of the plurality of subjects, and the compiling comprises organizing the sequence reads by the partition-specific barcodes and the plurality of target variants into a matrix of sequence reads, partitions, and subjects for further analysis.

One general aspect is directed to a method that includes: obtaining, by a data processing system, genotypes of multiple different loci for a plurality of subjects, where the different loci comprise a predetermined set of target variants that provide a unique variant combination associated with each subject from the plurality of subjects; obtaining, by the data processing system, sequence reads for nucleic acid from the plurality of subjects, where the sequence reads comprise nucleic acid sequences with associated partition-specific barcodes that identify a partition from which each sequence read originated; deconvoluting, by the data processing system, the sequence reads by the partition-specific barcodes to group the sequence reads by the partition from which each sequence read originated where the deconvoluting comprises organizing the sequence reads by the partition-specific barcodes into a matrix of sequence reads and partitions; determining, by the data processing system, a presence of a plurality of target variants in the sequence reads grouped into each of the partitions; determining, by the data processing system, whether the sequence reads grouped into each of the partitions originate from more than one subject from the plurality of subjects based on a first statistical comparison of the unique variant combination associated with each subject and the plurality of target variants in the sequence reads grouped into each of the partitions; when the sequence reads grouped into a partition do originate from more than one subject from the plurality of subjects, discarding, by the data processing system, the sequence reads associated with the partition; and when the sequence reads grouped into a partition do not originate from more than one subject from the plurality of subjects, assigning, by the data processing system, a subject from the plurality of subjects to the partition based on a second statistical comparison of the unique variant combination associated with the each subject from the plurality of subjects and the plurality of target variants in the sequence reads grouped into the partition. Other embodiments of this aspect include corresponding computer systems, apparatus, and computer programs recorded on one or more computer storage devices, each configured to perform the actions of the methods.

One general aspect is directed to a method that includes: isolating or encapsulating, by an expression profiling framework, target molecules in partitions with associated barcodes, where the target molecules are obtained from a plurality of subjects; subjecting, by the expression profiling framework, the partitions to conditions for enzymatic incorporation of the barcodes into nucleic acid of the target molecules or amplification products thereof; sequencing, by the expression profiling framework, the nucleic acid and barcodes to obtain sequence reads for the nucleic acid and associated barcodes that identify a partition from which each sequence read originated; obtaining, by the expression profiling framework, genotypes of multiple different loci for the plurality of subjects, where the different loci comprise a predetermined set of target variants that provide a unique variant combination associated with each subject from the plurality of subjects; determining, by the expression profiling framework, a presence of a plurality of target variants in each set of sequence reads having the same barcode; removing sets of sequence reads for partitions that are determined to be doublets based on a first statistical comparison of the unique variant combination associated with each subject and the plurality of target variants in each set of sequence reads having the same barcode; and assigning a subject from the plurality of subjects to each remaining set of sequence reads based on a second statistical comparison of the unique variant combination associated with the each subject from the plurality of subjects and the plurality of target variants in each set of sequence reads having the same barcode. Other embodiments of this aspect include corresponding computer systems, apparatus, and computer programs recorded on one or more computer storage devices, each configured to perform the actions of the methods.

Implementations may include one or more of the following features. The method where the first statistical comparison comprises generating a mixture model to calculate a likelihood that the set of sequence reads grouped into each partition originates from more than one subject from the plurality of subjects. The method where the second statistical comparison comprises: (i) determining a number of the sequence reads grouped into each of the partitions that overlap with the target variants of the unique variant combination associated with each subject, and (ii) determining a maximum likelihood that the set of sequence reads grouped into each of the partitions originated from each subject based on the determined number of the sequence reads grouped into each of the partitions that overlap with the target variants of the unique variant combination associated with each subject. The method where the variants from the set of target variants are single nucleotide polymorphisms (SNPs). The method further including compiling, by the expression profiling framework, a set of sequence data for each subject of the plurality of subjects, where the set of sequence data comprises the set of sequence reads grouped into each partition assigned to each subject of the plurality of subjects, and the compiling comprises organizing the sequence reads by the barcodes and the plurality of target variants into a matrix of sequence reads, partitions, and subjects for further analysis.

One general aspect is directed to a method of generating nucleotide sequence from multiple samples (e.g., from different individuals or sources). The method may comprise: forming a mixture of cells or nuclei in partitions wherein a majority of the partitions that have cells or nuclei contain only one cell, and wherein some partitions contain two or more cells or nuclei; in partitions, linking polynucleotides from the cells or nuclei to a partition-specific barcode and optionally a unique molecular identifier barcode; combining contents of a plurality (e.g., at least 10³, 10⁴, 10⁵, 10⁶, 10⁷ or more) of partitions into a bulk mixture; generating nucleotide sequencing reads from the bulk mixture, wherein the sequencing reads comprise reads of the linked unique molecular identifier barcode and partition-specific barcode; within sequencing reads having the same partition-specific barcode, determining the genotypes of a plurality of different target variants (e.g., at least 10, 20, 30, 40, 50, 60 75, or 100, e.g., 10-100, optionally wherein the target variants are SNPs); determining whether sequencing reads having the same partition-specific barcode contain sequence reads from more than one cell based on the genotypes (e.g., based on the unique variant combination in different cells, e.g., that come from different samples from different individuals (e.g., humans)), and discarding data comprising from droplets that contain more than one cell; deconvoluting sequences from the remaining droplets based on the unique variant combinations, where sequencing data from different cell types are sorted based on the determined genotypes. In some embodiments, the polynucleotides are genomic DNA, cDNA, or RNA. In some embodiments, the method involves Ribonucleic Acid analysis by Sequencing (RNA-seq), Assay for Transposase-Accessible Chromatin by Sequencing (ATAC-seq) (where nuclei (e.g., permeabilized nuclei) are used), Cellular Indexing of Transcriptomes and Epitopes by Sequencing (CITE-seq) (which involves RNA sequencing along with gaining quantitative and qualitative information on surface proteins with available antibodies on a single cell level), or a combination thereof. In some embodiments, the partitions are wells or droplets (e.g., in an emulsion).

In another aspect, the invention provides methods for multiplex analysis of samples from different individuals that involves pooling a plurality of samples from different individuals to produce a pooled sample and conducting a sequencing reaction on the pooled sample to produce a plurality of sequence reads. The method then involves associating each of the plurality of sequence reads back to the different individuals by identifying unique variants (e.g., single nucleotide polymorphisms (SNP's)) in each of the plurality of sequence reads and correlating the unique variants in each of the plurality of sequence reads to previously obtained sequence information of each different individual, wherein a highest match of the unique variants in each of the plurality of sequence reads to an individual's previously obtained sequence information determines which sequence read is associated with which different individual.

In certain embodiments conducting the sequencing reaction comprises generating a plurality of partitions, each partition comprising a single cell (e.g., immune cell) from a different individual, and sequencing nucleic acid from each single cell from each of the plurality of partitions to generate a plurality of sequence reads, wherein each of the plurality of sequence reads comprises a partition specific barcode. The method may then additionally involve associating each of the plurality of sequence reads back to one of the plurality of partitions based on the partition specific barcode. The skilled artisan will understand that in certain embodiments, the associating steps are performed by a data processing system operably associated with a sequencing instrument that generates the plurality of sequence reads.

In certain embodiments, duplicate sequence reads of the plurality of sequence reads are removed prior to the associating steps. The correlating step may further involve performing a statistical comparison of the unique variants with each different individual's previously obtained sequence information. For example, the statistical comparison may include: (i) determining a number of the plurality of sequence reads grouped into each of the partitions that overlap with each different individual's previously obtained sequence information, and (ii) determining a maximum likelihood that the plurality of sequence reads grouped into each of the partitions originated from an individual based on the determined number of the plurality of sequence reads grouped into each of the partitions that overlap with each different individual's previously obtained sequence information.

In certain embodiments, the first associating step comprises organizing the plurality of sequence reads by the partition-specific barcodes into a matrix of sequence reads and partitions. In certain embodiments, prior to the assigning, the method may involve determining whether the sequence reads grouped into each of the partitions originate from more than one individual from the different individuals. When the sequence reads grouped into a partition do originate from more than one individual from the different individuals, the sequence reads associated with the partition are removed from the matrix. When the sequence reads grouped into a partition do not originate from more than one individual from the different individuals, performing the assigning. In certain embodiments, the determining comprises generating a mixture model to calculate a likelihood that the sequence reads grouped into each partition originates from more than one individual from the different subjects.

Another aspect of the invention provides methods for assessing response to a treatment. The methods may involve pooling a first sample from a first individual and a second sample from a second individual to produce a pooled sample. The first sample comprises cells from the first individual that have received a treatment and cells from the first individual that have not received the treatment. The second sample comprises cells from the second individual that have received the treatment and cells from the second individual that have not received the treatment.

The method may then involve conducting a sequencing reaction on the pooled sample to produce a plurality of sequence reads, and associating each of the plurality of sequence reads back to the different individuals by identifying unique variants in each of the plurality of sequence reads and correlating the unique variants in each of the plurality of sequence reads to previously obtained sequence information of each different individual, wherein a highest match of the unique variants in each of the plurality of sequence reads to an individual's previously obtained sequence information determines which sequence read is associated with which different individual. The method may then involve establishing a first expression profile for the first individual from the sequence reads associated with the first individual, wherein the first expression profile shows expression differences between the cells of the first individual that received the treatment and the cells of the first individual that did not receive the treatment, and establishing a second expression profile for the second individual from the sequence reads associated with the second individual, wherein the second expression profile shows expression differences between the cells of the second individual that received the treatment and the cells of the second individual that did not receive the treatment.

In certain embodiments conducting the sequencing reaction comprises generating first and second set of partitions, wherein the first set of partitions comprises partitions comprising single cells from the first individual that have received a treatment and partitions comprising single cells from the first individual that have not received the treatment, and wherein the second set of partitions comprises partitions comprising single cells from the second individual that have received the treatment and partitions comprising single cells from the second individual that have not received the treatment; and sequencing nucleic acid from the first and second set of partitions to generate a plurality of sequence reads, wherein each of the plurality of sequence reads comprises a partition specific barcode. The methods may additionally involve associating each of the plurality of sequence reads back to one of the first or second sets of partitions based on the partition specific barcode.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

FIG. 1A depicts a pipeline for experimental multiplexing of unrelated individuals, loading onto droplet-based single-cell RNA-sequencing instrument, and computational demultiplexing (demux) and doublet removal using demuxlet in accordance with various embodiments.

FIG. 1B depicts assuming equal mixing of eight individuals, four genetic variants can recover the sample identity of a cell in accordance with various embodiments.

FIG. 1C depicts 87.5% of doublets will contain cells from two different samples.

FIG. 2 depicts the probability of a given number of SNPs that can uniquely assign a cell correctly to its donor given the number of samples multiplexed. The probability was calculated over 100 simulated cells. Error bar determined by 10 simulation replicates.

FIG. 3 depicts a number of usable singlets (triangle) and total number of cells sequenced (circle) as a function of number of individuals multiplexed at a fixed undetected doublet rate estimated from 1000 cells (1% -10%).

FIG. 4 depicts a cost per singlet (triangle) and per all cells (circle) as a function of number of individuals multiplexed at a fixed undetected doublet rate estimated from 1000 cells (1% -10%).

FIG. 5 depicts a flowchart illustrating a process for droplet-based sequencing to obtain sequence reads for nucleic acid from a plurality of subjects in accordance with various embodiments.

FIG. 6 depicts a flowchart illustrating a process for multiplexed droplet-based sequencing using natural genetic barcodes in accordance with various embodiments.

FIG. 7 depicts a block diagram of an expression profiling framework in accordance with various embodiments

FIG. 8 depicts a block diagram of a computing system or data processing system in accordance with various embodiments

FIG. 9 depicts detection boundaries of likelihood differences between mixture model for doublets (LLK12) and singlets (SNG.LLK1).

FIG. 10 depicts a probability to demultiplex up to 64 unrelated individuals versus number of reads overlapping SNPs.

FIG. 11 depicts probability and error rates to detect doublets from up to 64 unrelated individuals versus number of reads overlapping SNPs. Dashed lines designate the theoretical limit for doublet detection OM-N)).

FIG. 12 depicts a number of SNPs detected per cell (y-axis) versus number of UMIs (x-axis).

FIG. 13 depicts a proportion of correctly assigned cells as a function of reads overlapping SNPs for 8 related individuals from 1000 Genomes. Circles represent averages over 6,145 cells downsampled to a total of 5,000, 10,000, 25,000, 50,000, 75,000 or 90,000 reads overlapping SNPs over all individuals.

FIG. 14A depicts an experimental design for equimolar pooling of cells from eight unrelated samples (S1-S8) into three wells (W1-W3). W1 and W2 contain cells from two disjoint sets of four individuals. W3 contains cells from all eight individuals.

FIG. 14B depicts demultiplexing singlets in each well recovers the expected individuals.

FIG. 14C depicts estimates of doublet rates versus previous estimates from mixed species experiments.

FIG. 14D depicts cell type identity determined by prediction to previously annotated peripheral blood mononuclear cell (PBMC) data.

FIG. 14E depicts a t-SNE plot of two individuals (Si and S5) from different wells are qualitatively concordant.

FIG. 15 depicts cell-type specific expression of single cells versus bulk. Average expression over single cells for each cell type (y-axis) versus the bulk expression of B cells, CD4+ T cells and monocytes (x-axis). Single cell averages were estimated for 8 individuals. Bulk expression was generated for 6 individuals.

FIG. 16 depicts estimates of cell type proportion from three well experiment of unstimulated PBMCs. Estimates of cell type proportion for wells W1 and W2 (x-axis) versus estimates of cell type proportion for the same individuals in well W3 (y-axis).

FIG. 17A depicts TSNE plot for wells W1, W2 and W3 separately.

FIG. 17B depicts QQ plot comparing differential expression analysis between W1 and W2 with W3 for the same individuals.

FIG. 18 depicts estimated versus observed doublet rates for unstimulated cells. Expected doublet rates for each pair of individuals (i,j) are calculated as pi*pj, where pi and pj are the proportion of singlets individuals i and j.

FIG. 19 depicts detected doublets from the IFN-beta stimulation experiment. Red (confidently called doublets). Pink (ambiguous). Gray (singlets).

FIG. 20A depicts t-SNE plot of unstimulated (blue) and IFN-β-stimulated (red) PBMCs and the presumed cell types. cM, CD14+CD16—monocytes; ncM, CD14+CD16+ monocytes; DC, dendritic cells; Mkc, megakaryocytes; Th, CD4+ T cells; B, B cells; Tc, CD8+ T cells; NK, natural killer cells.

FIG. 20B depicts cell-type-specific expression in stimulated (left) and unstimulated (right) cells. Differentially expressed genes shown (FDR<0.05, |log(FC)|>1). Each column represents cell-type-specific expression for each individual from demuxlet.

FIG. 20C depicts observed variance (y axis) in mean expression over all PBMCs from each of the eight individuals versus expected variance (x axis) over synthetic replicates sampled across all cells (light blue, pink) or replicates matched for cell type proportion (blue, red).

FIG. 20D depicts cell type proportions for each individual in unstimulated and stimulated cells.

FIG. 20E depicts a correlation between sample replicates in control and stimulated cells.

FIG. 20F depicts a number of significantly variable genes in each cell type and condition.

FIG. 21 depicts estimates of cell type proportion from unstimulated and IFN-beta stimulated PBMCs. Estimates of cell type proportion for unstimulated cells (x-axis) versus stimulated cells (y-axis) for 8 individuals.

FIG. 22 depicts scatter plot of estimates of cell type proportion from dscRNA-seq (x-axis) and FACS sorting (y-axis) for 6 lupus patients after IFN-beta stimulation.

FIG. 23 depicts gene expression correlation between dscRNA-seq data and ImmGen mouse microarray data. Average abundance is calculated for each cell-type by stimulation. 2000 homologous genes are used.

FIG. 24 depicts a correlation of foldChange before and after IFN-beta stimulation of 1000 expressed genes between published CD4 microarray (Ye et al., Science) and monocyte derived dendritic cells RNA-seq (Ye et al., unpublished) (y-axis) with dscRNA-seq cell type specific response (x-axis).

FIG. 25 depicts IFN-beta fold change correlation between dscRNA-seq data (y axis) and qPCR data from CD4+ T cells (x axis).

FIG. 26 depicts expected versus observed variability in cell populations. Variance of average expression over single cells across 8 individuals (y) versus variance of average expression over single cells across 8 synthetic replicates matching cell number for each individual (x).

FIG. 27A depicts observed variance (y axis) in mean expression over all PBMCs from each individual versus expected variance (x axis) over synthetic replicates sampled across batch 1 (left, N=8) and batch 3 (right, N=15).

FIG. 27B depicts an association of chr10:3791224 with NK cell type proportions.

FIG. 27C depicts genome-wide and chromosome six Manhattan plots across all major cell types. Horizontal lines correspond to FDR<0.1 (blue) and FDR<0.05 (red).

FIG. 27D depicts Q-Q plots across all genes and subsets of previously published eQTLs in relevant cell types are shown for B, cM, and Tx populations.

FIG. 27E depicts notable cis-eQTLs across all major immune cell types are marked with *(FDR<0.25), **(FDR<0.1), and ***(FDR<0.05). Lack of association is marked with NS (not significant).

FIG. 28 depicts observed variability in gene expression is correlated between batches.

FIGS. 29A and 29B depict the umber of cells demultiplexed for each individual through demuxlet in FIG. 29A.) low nuclei concentration and in FIG. 29B.) high nuclei concentration. Four individuals were included in the experiment and Four additional genotype profiles were added to demuxlet to assess the false positive rate.

FIG. 30A depicts a CITE-seq workflow in accordance with various embodiments.

FIG. 30B depicts RNA and protein levels for CD3 from 15 demultiplexed individuals.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

I. Introduction

In various embodiments, techniques are provided (e.g., a method, a system, non-transitory computer-readable medium storing code or instructions executable by one or more processors) for multiplexed droplet-based sequencing using natural genetic barcodes. Droplet-based sequencing has enabled rapid, massively parallel profiling of molecular content of cells. However, assessing inter-individuals variation (differential expression and patient subtyping for example) has been hampered by costly and inefficient sample processing and technical batch effects.

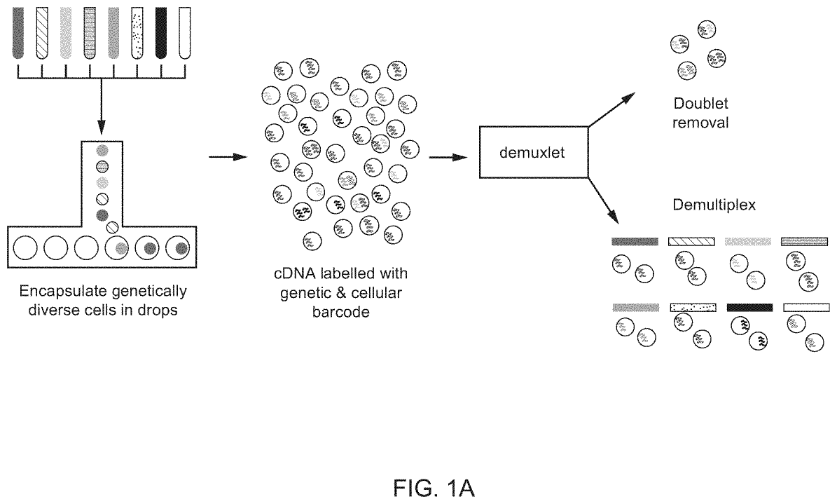

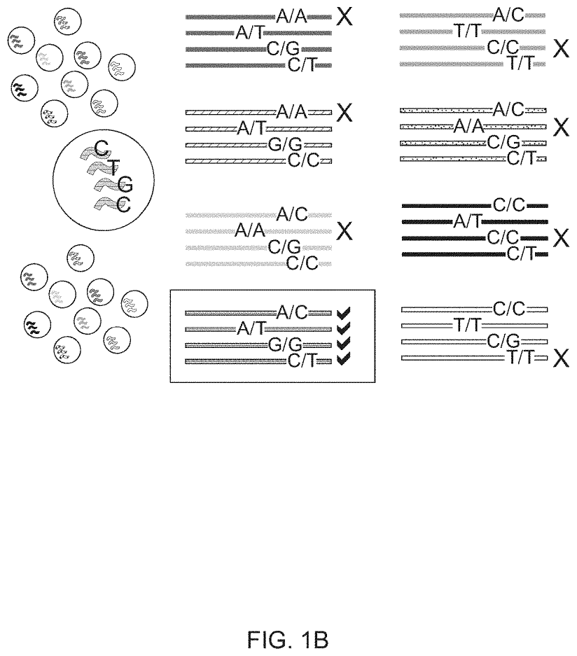

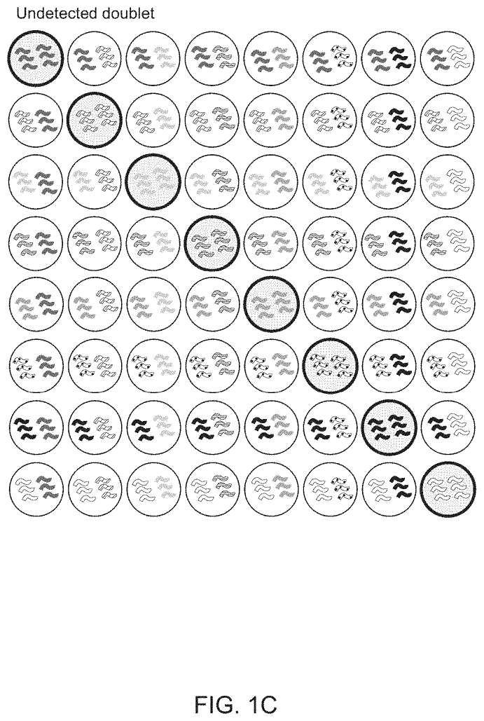

Various embodiments disclosed herein are directed to a computational tool or technique, demuxlet, that harnesses natural genetic variation to determine the sample identity of each partition containing a single target molecule such as a cell (singlet) and detect partitions containing two or more target molecules (doublets or multiplet)(see, e.g., FIG. 1A). These capabilities enable multiplexed droplet-based sequencing in which molecular targets from unrelated individuals are pooled, loaded in higher concentrations (e.g., greater than 20,000 cells in a single test run) into an expression profiling framework, and captured at higher throughput than in standard workflows.

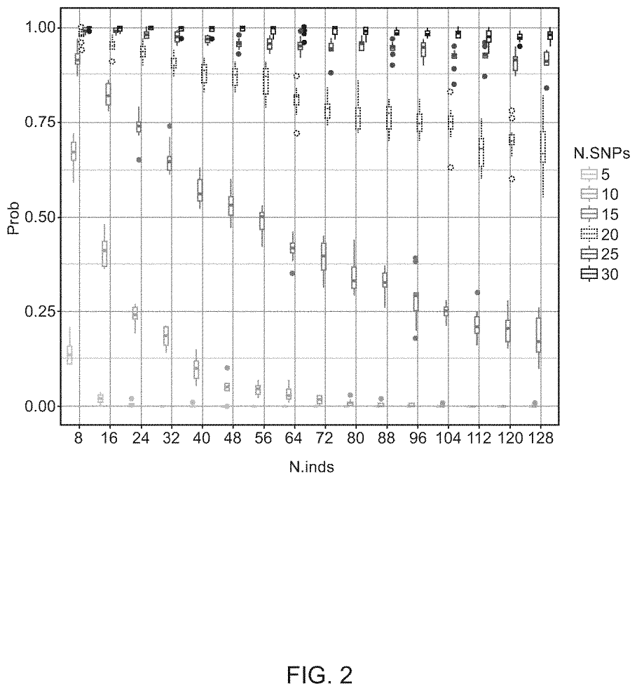

Demuxlet implements a statistical model for evaluating the likelihood of observing sequence reads (e.g., RNA-seq reads, genomic DNA reads, ATAC-seq reads, CITE-seq reads, etc.) overlapping a unique variant combination (e.g., a set of single nucleotide polymorphisms (SNPs)) from each target molecule containing partition (e.g., droplet). Given a set of best-guess genotypes or genotype probabilities obtained from genotyping, imputation or sequencing, demuxlet uses maximum likelihood to determine the most likely donors for each partition using a mixture model. A small number of reads overlapping common variants is sufficient to accurately identify each partition. For example, given a pool of eight individuals and a set of uncorrelated SNPs, each with 50% minor allele frequency (MAF), four reads overlapping SNPs are sufficient to uniquely assign a singlet to the donor of origin (see, e.g., FIG. 1B) and twenty reads overlapping SNPs can distinguish every sample with >98% probability in simulation (see, e.g., FIG. 2). Accordingly, an expression profiling framework running demuxlet is capable of distinguishing not only between target molecules but also between subjects, and the pooling of target molecules from different subjects in a single test run advantageously results in lower per-sample library preparation cost and eliminates confounding effects.

One illustrative embodiment of the present disclosure is directed to a technique that comprises: obtaining, by a data processing system, genotypes of multiple different loci for a plurality of subjects, where the different loci comprise a predetermined set of target variants that provide a unique variant combination associated with each subject from the plurality of subjects; obtaining, by the data processing system, sequence reads for nucleic acid from the plurality of subjects, where the sequence reads comprise nucleic acid sequences with associated partition-specific barcodes that identify a partition from which each sequence read originated; deconvoluting, by the data processing system, the sequence reads by the partition-specific barcodes to group the sequence reads by the partition from which each sequence read originated; determining, by the data processing system, a presence of a plurality of target variants in the sequence reads grouped into each of the partitions; and assigning, by the data processing system, at least one subject from the plurality of subjects to one or more partitions based on a first statistical comparison of the unique variant combination associated with the each subject from the plurality of subjects and the plurality of target variants in the sequence reads grouped into the one or more partitions.

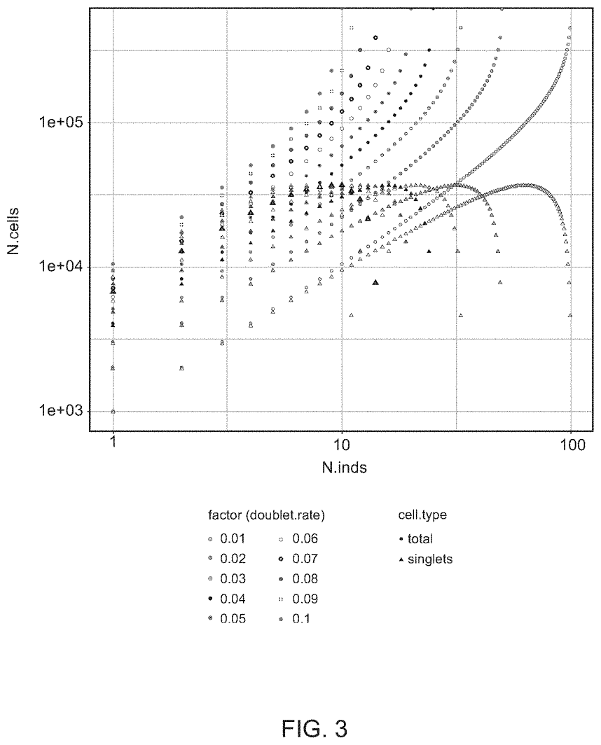

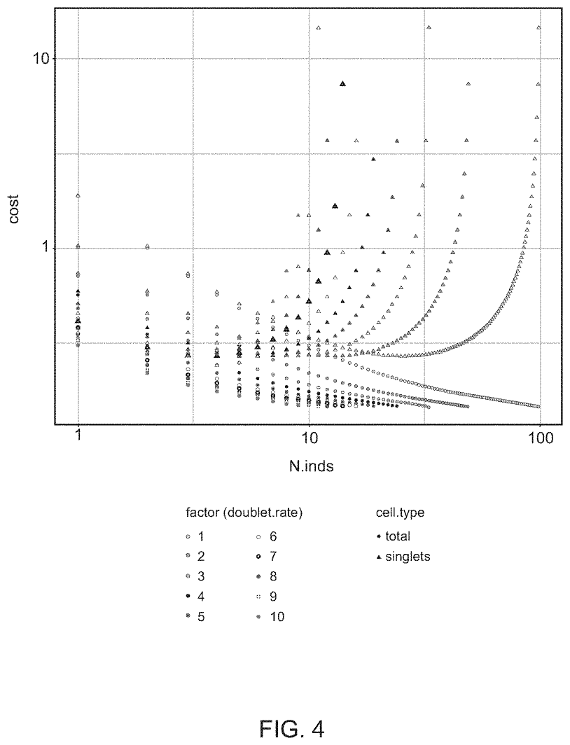

However, by multiplexing target molecules from even a small number of individual subjects, the probability that a doublet contains target molecules from different individuals is very high (1−1/N, for example, 87.5% for N=8 samples) (see, e.g., FIG. 1C). For example, if a 1,000-cell run without multiplexing results in 990 singlets with a 1% undetected doublet rate, multiplexing 1,570 cells each from 63 samples can theoretically achieve the same rate of undetected doublets, producing up to 37-fold more singlets (36,600) if the sample identity of every droplet can be perfectly demultiplexed (see, e.g., FIG. 3). Profiling 30,000 cells multiplexed from 26 individuals can theoretically generate 23-fold more singlets at the same effective doublet rate, minimizing the effects of sequencing doublets (see, e.g., FIG. 4). To identify doublets for removal prior to profile analysis, demuxlet may also implement a mixture model to calculate the likelihood that the sequence reads originated from two individuals, and the likelihoods are compared to determine whether a partition contains target molecules from one or multiple subjects (e.g., two subjects). Accordingly, an expression profiling framework running demuxlet is capable of detecting multiple target molecules from different subjects, and advantageously the pooled target molecules could be loaded at higher concentrations, enabling additional reduction in per-cell library preparation cost.

Another illustrative embodiment of the present disclosure is directed to a technique that comprises: obtaining, by a data processing system, genotypes of multiple different loci for a plurality of subjects, where the different loci comprise a predetermined set of target variants that provide a unique variant combination associated with each subject from the plurality of subjects; obtaining, by the data processing system, sequence reads for nucleic acid from the plurality of subjects, where the sequence reads comprise nucleic acid sequences with associated partition-specific barcodes that identify a partition from which each sequence read originated; deconvoluting, by the data processing system, the sequence reads by the partition-specific barcodes to group the sequence reads by the partition from which each sequence read originated; determining, by the data processing system, a presence of a plurality of target variants in the sequence reads grouped into each of the partitions; determining, by the data processing system, whether the sequence reads grouped into each of the partitions originate from more than one subject from the plurality of subjects based on a first statistical comparison of the unique variant combination associated with each subject and the plurality of target variants in the sequence reads grouped into each of the partitions; when the sequence reads grouped into a partition do originate from more than one subject from the plurality of subjects, discarding, by the data processing system, the sequence reads associated with the partition; and when the sequence reads grouped into a partition do not originate from more than one subject from the plurality of subjects, assigning, by the data processing system, a subject from the plurality of subjects to the partition based on a second statistical comparison of the unique variant combination associated with the each subject from the plurality of subjects and the plurality of target variants in the sequence reads grouped into the partition.

II. Techniques for Droplet-Based Sequencing

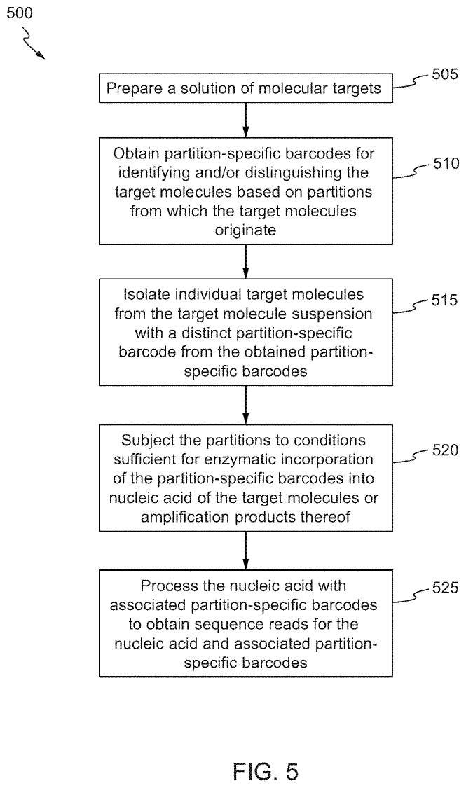

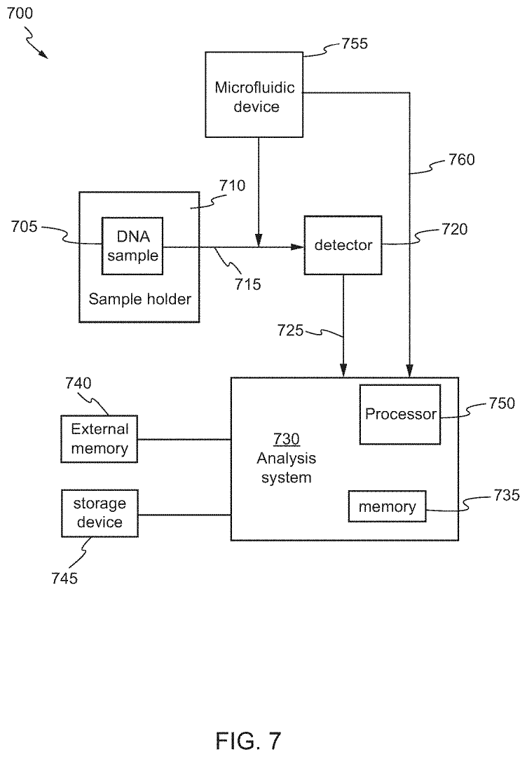

FIG. 5 shows a flowchart 500 that illustrates a process for droplet-based sequencing to obtain sequence reads for nucleic acid from a plurality of subjects. In some embodiments, the processes depicted in flowchart 500 may be implemented by the architecture, systems, and techniques that are part of an expression profiling framework depicted in FIG. 7. At step 505, a solution of molecular targets is prepared. In various embodiments, the molecular targets are obtained from a plurality of subjects and placed into solution. In some embodiments, the molecular targets are cells, nuclei, viruses, or other form of nucleic acid. The molecular targets may be obtained from each of the subjects by one or more means including the dissociation of a complex tissue such as brain tissue into individual molecular targets or the isolation of molecular targets from a biological fluid such as blood. At step 510, partition-specific barcodes are obtained for identifying and/or distinguishing the target molecules based on partitions from which the target molecules originate. In some embodiments, the partition-specific barcodes are nucleic acid barcode sequences, e.g., about 5 to 20 bases in length. In some embodiments, the partition-specific barcodes are provided on the surface of a microparticle or encapsulated within a microparticle. For example, each microparticle may contain more than 10⁵ oligonucleotide primers that comprise a polymerase chain reaction (PCR) handle (e.g., a universal sequence that will be common to amplicons and can be used by common primers in a later step), a partition-specific barcode, optionally a unique molecular identifier (UMI), and a 3′ sequence that will hybridize to target polynucleotides in the sample. The PCR handle may be a constant sequence of nucleotides that is identical on all primers and microparticles, which allows PCR amplification after the handle is attached to a nucleic acid sequence of the target molecule. The partition-specific barcode is only identical across all the primers of a same microparticle but different from partition-specific barcodes on other beads, and thus allows the recovery of the partition of origin for each target molecule. The UMIs are different on each primer and allow for sequence reads to be digitally counted and to identify PCR duplicates. The oligonucleotide is different or the same on each primer for capturing and priming the read of a target nucleic acid, for example, a 30-bp oligodT sequence may be used on all primers to capture mRNA and prime reverse transcription.

At step 515, individual target molecules (e.g., a cell) from the target molecule suspension (e.g., a cell suspension) are isolated with a distinct partition-specific barcode from the obtained partition-specific barcodes. In some embodiments, individual target molecules are isolated together with partition-specific barcoded microparticles, using a microfluidic device e.g., the microfluidic device shown and discussed with respect to FIG. 7). For example, the microfluidic device may join two aqueous flows into discrete partitions. One flow contains the target molecule suspension, and the other flow contains the partition-specific barcoded microparticles in a lysis buffer. The discrete “partitions” used herein refers to objects, such as droplets, including multiple emulsions (such as double emulsions), wells, compartments and containers capable of encapsulating and/or containing one or more molecular targets (e.g., a cell, nucleus, virus, etc.) as described herein and a partition-specific barcode such as a partition-specific barcoded microparticle as described herein.

At step 520, the partitions are subjected to conditions sufficient for enzymatic incorporation of the partition-specific barcodes into nucleic acid of the target molecules or amplification products thereof. In some embodiments, the nucleic acid from the target molecule hybridizes with the partition-specific barcode in each partition. Hybridization involves the annealing of one nucleic acid to another, complementary nucleic acid, i.e., a nucleic acid having a complementary nucleotide sequence. In some embodiments, in which the target molecule is a cell, following isolation of the cell in the partition with the partition-specific barcode, the cell is lysed releasing nucleic acid, and thus allowing for the enzymatic incorporation of the partition-specific barcodes into the nucleic acid. As used herein, “nucleic acid”, or “nucleic acid molecule” refers to a polymeric form of nucleotides of any length, either deoxyribonucleotides or ribonucleotides, or analogs thereof. The terms encompass, e.g., DNA, RNA and modified forms thereof. Polynucleotides may have any three-dimensional structure, and may perform any function, known or unknown. Non-limiting examples of polynucleotides include a gene, a gene fragment, exons, introns, mRNA, transfer RNA, ribosomal RNA, ribozymes, cDNA, recombinant polynucleotides, branched polynucleotides, plasmids, vectors, isolated DNA of any sequence such as genomic DNA, control regions, isolated RNA of any sequence, nucleic acid probes, and primers.

At step 525, the nucleic acid with associated partition-specific barcodes are processed to obtain sequence reads for the nucleic acid and associated partition-specific barcodes. In various embodiments, the sequence reads comprise nucleic acid sequences with associated partition-specific barcodes that identify the partition from which each sequence read originated. The processing may comprise one or more steps including reverse transcription, amplification, and/or sequencing. The sequence reads may be obtained and analyzed using any suitable sequencing technique, as described in detail in Section IV. For example, to sequence the transcriptomes of individual cells a variety of workflows can be used. In one workflow (e.g., where RNA sequences are to be determined), reagents sufficient for cDNA synthesis and amplification of the cell transcriptome, such as SMART™ reagents (available from Clontech Laboratories, Palo Alto, Calif.—see, e.g., Zhu et al., BioTechniques 30:892-897 (2001)), can be introduced into a partition containing cell lysate together with reagents for partition-specific barcoding. Then, by thermally cycling the partitions, the reactions can be performed in the same step, resulting in cDNA synthesis of the mRNA transcriptomes, their amplification, and tagging of the ends of the amplification products with partition-specific barcodes using, for example, splicing by overlap extension PCR (SOEing PCR).This is a relatively straightforward workflow that can be performed on a small number of devices, or potentially a single device, and provides information about the ends of the transcripts, which is useful for expression profiling.

Alternatively, the cDNA synthesis, which relies on reverse transcriptase, can be performed in one step, and then reagents can be added to perform the amplification and partition-specific barcoding in later steps. Allowing these reactions to be performed in different steps, allows for the modification of buffers after the individual steps, which could be valuable for optimizing the reactions to obtain the most accurate data. Both of the described methods provide sequencing reads for the ends of the mRNA transcripts. If it is desirable to obtain the full transcript sequence, then there are also several options. One option is to obtain the partition-specific barcoded transcripts from the previously described approaches and sequence them with a long molecule sequencing technology such as, for example, the PACBIO RS II sequencer, Pacific Biosciences, Menlo Park, Calif. Alternatively, the long molecule sequencing method described herein can also be used for this purpose. Both of these methods have the advantage of not only providing reads, which relate splice variation, but also allow the reconstitution of full length individual transcript molecules.

An alternative approach is to perform fragmentation and barcoding as follows. The cell mRNA is reverse transcribed into cDNA and amplified in one or two steps, in which cDNA synthesis occurs first and reagents sufficient for amplification are added in a second step. The amplified molecules are then subjected to fragmentation using any suitable method/reagents, for example, Fragmentase® or transposase, and partition-specific barcodes are then introduced with any of the previously described methods, such as ligation or SOEing using adaptors that are inserted during, for example, fragmentation with transposase. In addition, the target molecules can be labeled with unique molecular identifiers (UMIs) before, during, or after the cDNA synthesis step. These UMIs are substantially distinct from one another and label the target molecules, allowing for more accurate transcript counting by taking advantage of the UMI diversity.

Yet another approach is to target specific transcripts for sequencing, which can be accomplished using a number of techniques. For example, specific primers can be used to reverse transcribe only certain sequences during the cDNA synthesis or amplification steps, which can then be subjected to partition-specific barcoding. This can be used to, for example, target the B or T cell receptor genes for sequencing in a cell population, which could be useful for identifying disease biomarkers or therapeutic antibodies. Other combinations can also be selected by multiplexing the primer sets to, for example, correlate the expression and sequences of multiple genes. Viral genes, for example, in HIV infection, can be correlated with the expression of host genes by designing primer sets that barcode only these genes. This has the advantage of providing much simpler data and also allowing the sequencing to be targeted to the genes of interest, which is useful in some applications of the present disclosure.

While sequencing of RNA (via cDNAs) are described herein, it should be noted the method can be adapted for other types of partition-based sequencing in which single cells or nuclei are introduced into separate partitions and assigned a partition-specific barcode. As an example, chromatin state can be investigated using ATAC-seq or single cell ATAC-seq (sc ATAC-seq) in which the status of chromatin is reflected in the abundance of sequencing reads from a certain location in chromatin. Partitions having more than one nuclei can be de-duplexed using the methods described herein. Similarly, or in combination, partitions having nuclei from different sources (individuals) can be identified using the method described herein. As another example, surface protein levels can be investigated using CITE-seq in which quantitative and qualitative information on surface proteins is reflected in the abundance of sequencing reads from the transcriptome as well as the proteome of single cells.

III. Techniques for multiplexed droplet-based sequencing using natural genetic barcodes

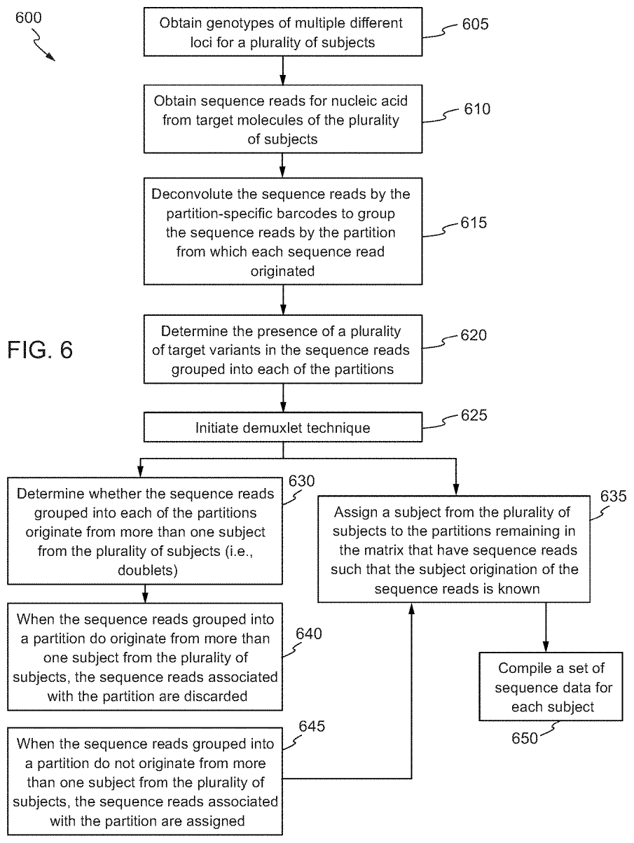

FIG. 6 illustrates processes and operations for multiplexed droplet-based sequencing using natural genetic barcodes. Individual embodiments may be described as a process which is depicted as a flowchart, a flow diagram, a data flow diagram, a structure diagram, or a block diagram. Although a flowchart may describe the operations as a sequential process, many of the operations may be performed in parallel or concurrently. In addition, the order of the operations may be re-arranged. A process is terminated when its operations are completed, but could have additional steps not included in a figure or the description thereof. A process may correspond to a method, a function, a procedure, a subroutine, a subprogram, etc. When a process corresponds to a function, its termination may correspond to a return of the function to the calling function or the main function.

The processes and/or operations depicted in FIG. 6 may be implemented in software (e.g., code, instructions, program) executed by one or more processing units (e.g., processors cores), hardware, or combinations thereof. The software may be stored in a memory (e.g., on a memory device, on a non-transitory computer-readable storage medium). The particular series of processing steps in FIG. 6 is not intended to be limiting. Other sequences of steps may also be performed according to alternative embodiments. For example, in alternative embodiments the steps outlined herein may be performed in a different order. Moreover, the individual steps illustrated in FIG. 6 may include multiple sub-steps that may be performed in various sequences as appropriate to the individual step. Furthermore, operations or steps may be added or removed depending on the particular applications. One of ordinary skill in the art would recognize many variations, modifications, and alternatives.

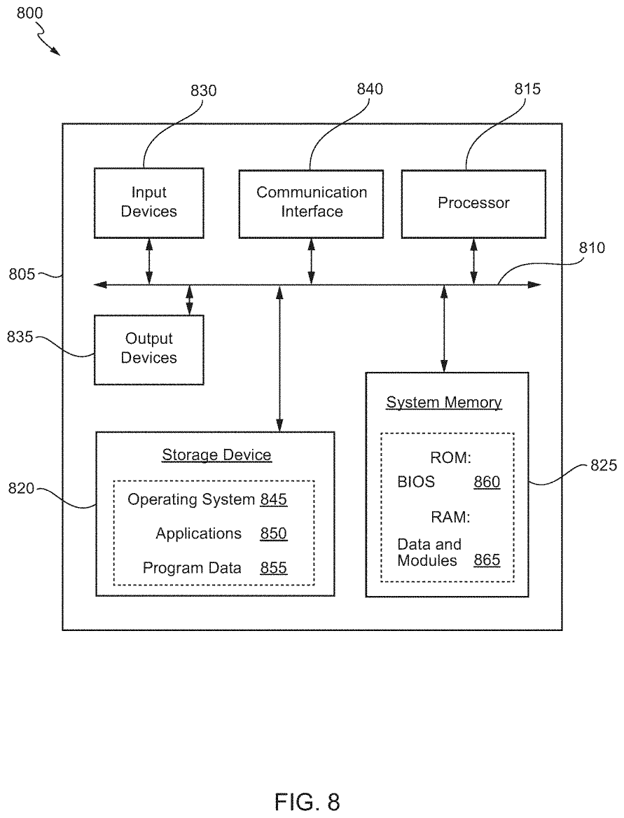

FIG. 6 shows a flowchart 600 that illustrates a process for multiplexed droplet-based sequencing using natural genetic barcodes. In some embodiments, the processes depicted in flowchart 600 may be implemented by the architecture, systems, and techniques depicted in FIGS. 7 and 8. At step 605, genotypes of multiple different loci are obtained for a plurality of subjects. The different loci comprise a predetermined set of target variants that provide a unique variant combination (i.e., a natural genetic barcode) associated with each subject from the plurality of subjects. In some embodiments, the variants are exonic variants. The genotypes may be obtained and analyzed using any suitable genotyping technique, as described in detail in Section IV. In some embodiments, one or more samples are obtained (e.g., by drawing blood from a subject), and genotyped, by a sequence analytical system, using a genotyping technique such as restriction fragment length polymorphism identification (RFLPI), random amplified polymorphic detection (RAPD) of genomic DNA, amplified fragment length polymorphism detection (AFLPD), polymerase chain reaction (PCR), DNA sequencing, allele specific oligonucleotide (ASO) probes, and hybridization to DNA microarrays or beads.

In some embodiments, the genotypes are obtained as an index or table of data comprising the genotype of each subject across the set of target variants. As should be understood, the genotype of each subject across the set of target variants will be different for each subject, and thus, will be used as a natural genetic barcode for each subject in subsequent processing. As used herein, a “variant” or “variant form” is a form or version of a gene or sequence of nucleotides that differs in some respect from other forms of the same gene or sequence of nucleotides, or from a reference gene or sequence. Examples of variants include: (i) single nucleotide polymorphisms (SNPs), which are DNA sequence variations that occur when a single nucleotide differs from the reference sequence. (ii) insertions, which occur when additional nucleotides are inserted in a sequence, relative to the reference sequence, (iii) deletions, which occur when there are missing nucleotides, relative to the reference sequence, (iv) substitutions which occur when multiple nucleotides are altered from the reference sequence, and (v) structural variants, which are changes where large sections of a chromosome or even whole chromosomes are duplicated, deleted or rearranged in some manner. In some embodiments, the set of target variants include more than three variants, for example four variants, eight variants, or at least fifty variants. In some embodiments, the target variants are SNPs. In some embodiments, the set of target variants includes at least 10, 20, 30, 40, 50, 60 75, or 100, e.g., 10-100, variants, optionally the target variants are SNPs.

At step 610, sequence reads are obtained for nucleic acid from target molecules of the plurality of subjects. The sequence reads comprise nucleic acid sequences with associated partition-specific barcodes that identify a partition from which each sequence read originated. In various embodiments, the sequence reads are obtained by droplet-based sequencing, as described with respect to FIG. 5. Droplet-based sequencing use microfluidics to compartmentalize the target molecules such as cells into nanoliter-sized reaction chambers or partitions for analysis of their nucleic acid while remembering the nucleic acids' target molecule of origin, using a partition-specific barcode strategy. This technique allows for quickly profiling thousands of individual target molecules and the creation of molecular atlases of gene expression for known target molecule classes and novel candidate target molecule subtypes.

At step 615, the sequence reads are deconvoluted by the partition-specific barcodes to group the sequence reads by the partition from which each sequence read originated. In some embodiments, the sequence reads are organized by their partition-specific barcodes into a matrix of sequence reads and partitions for further analysis. At step 620, the presence of a plurality of target variants is determined in the sequence reads grouped into each of the partitions. In some embodiments, the sequence reads are queried for a plurality of target variants of the predetermined set of target variants. In some embodiments, the query includes searching the sequence reads for evidence of the target variants from the predetermined set of target variants and calling or identifying the plurality of target variants in the sequence reads. The sequence reads may be queried using any suitable variant calling technique. For example, techniques may include the alignment of short reads and application of various statistical algorithms and models such as Bayesian models, Probabilistic models, Fisher's Exact Test, and string graphs to call or identify the one or more variants in the sequence reads. These algorithms and models specialize in calling or identifying discordant sequences in the ever-changing heterogeneous target molecules. In some embodiments, a deep read depth >500× is preferred to discern true variants from artifacts. In some embodiments, the plurality of target variants determined in the sequence reads grouped into each of the partitions are added to the matrix of sequence reads and partitions for further analysis.

At step 625, a sample demultiplexing and multiplet detection technique (demuxlet) is initiated for the sequence reads in the matrix to: (i) remove sequence reads from the matrix for partitions that are determined to be doublets (i.e., comprising sequence reads from two target molecules)(see, e.g., step 630), and (ii) assign a subject from the plurality of subjects to the partitions remaining in the matrix that have sequence reads such that the subject origination of the sequence reads is known (see, e.g., step 635). At step 630, whether the sequence reads grouped into each of the partitions originate from more than one subject from the plurality of subjects (i.e., doublets) is determined based on a first statistical comparison of the unique variant combination associated with each subject and the plurality of target variants in the sequence reads grouped into each of the partitions.

In various embodiments, to identify doublets, the demuxlet technique uses a mixture model (e.g., first statistical comparison) to calculate the likelihood that the sequence reads grouped into a partition originates from two individuals, and the likelihoods are compared to determine whether a droplet contains target molecules from one or two samples. If sequence reads from the c-th droplet originate from two different samples, S₁, S₂ with mixing proportions (1-α) : α, then the likelihood in (1) can be represented as the following mixture distribution:

L_{c}(s₁, s₂, α)=πᵥ₌₁^{V}[Σ_{g1,g2}{πᵢ₌₁^{d}^{_{cv}}(Σₑ₌₀¹(1−α)Pr(b_{cvi}|g₁, e)+αPr(b_{cvi}|g₂, e))Pₛᵥ^{(g1)}Pₛᵥ^{(g2)}}] To reduce the computational cost, certain embodiments utilize discrete values of: α ∈{α₁, . . . , α_{M}}, (e.g. 5-50% by 5%). It is possible determine that a partition is a doublet between samples s₁, s₂ if and only if

$$\frac{{max}_{{s}_{1} , {s}_{2} , α} {L}_{c} ( {s}_{1} , {s}_{2} , α )}{{max}_{s} {L}_{c} ( s )} \geq t$$

and the most likely mixing proportion is estimated to be argmaxₐL_{c}(s₁, s₂, α). It is possible to determine that the partition contains sequence reads from only a single individual s (i.e., a singlet) if

$$\frac{{max}_{{s}_{1} , {s}_{2} , α} {L}_{c} ( {s}_{1} , {s}_{2} , α )}{{max}_{s} {L}_{c} ( s )} \leq \frac{1}{t} .$$

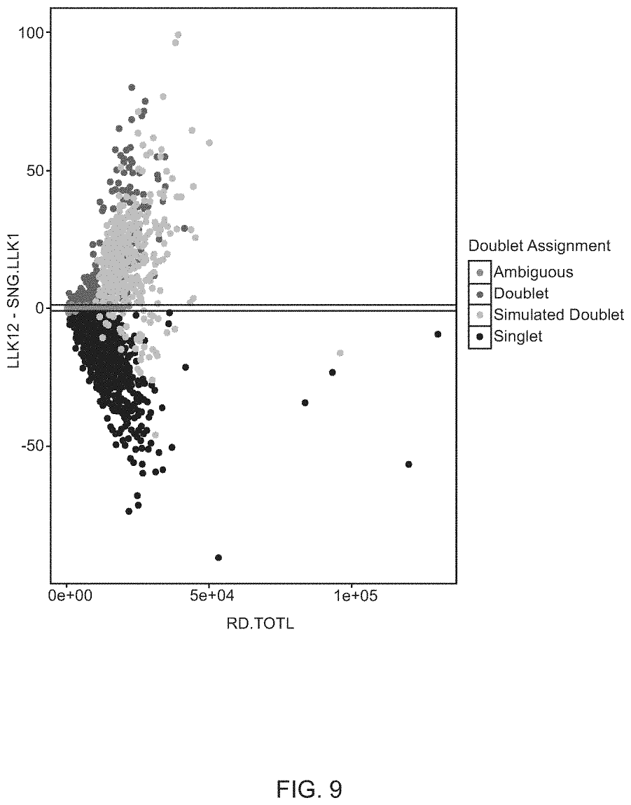

The less confident partitions, may be classified as ambiguous. FIG. 9 illustrates the distribution of singlet, doublet likelihoods and the decision boundaries when t=2 was used.

At step 640, when the sequence reads grouped into a partition do originate from more than one subject from the plurality of subjects, the sequence reads associated with the partition are discarded. For example, the sequence reads are removed from the matrix for partitions that are determined to be doublets. In certain embodiments, when the sequence reads grouped into a partition do originate from more than one subject from the plurality of subjects or it is ambiguous as to whether the sequence reads grouped into a partition do originate from more than one subject from the plurality of subjects, the sequence reads associated with the partition are discarded. At step 645, when the sequence reads grouped into a partition do not originate from more than one subject from the plurality of subjects, a subject from the plurality of subjects is assigned to the partition based on a second statistical comparison of the unique variant combination associated with each subject from the plurality of subjects and the plurality of target variants in the sequence reads grouped into the partition. At step 635, the sequence reads remaining for each partition in the matrix are assigned a subject from the plurality of subjects based on a second statistical comparison of the unique variant combination associated with each subject from the plurality of subjects and the plurality of target variants in the sequence reads grouped into the partition.

In various embodiments, to infer the sample identity for each partition, the demuxlet technique uses a statistical model (e.g., second statistical comparison) for evaluating the likelihood of observing sequence reads overlapping the unique variant combination associated with the each subject from the plurality of subjects. Given a set of best-guess genotypes or genotype probabilities obtained from genotyping, imputation or sequencing, the demuxlet technique uses maximum likelihood to determine the most likely donors for each droplet using a mixture model. For example, consider sequence reads from C barcoded droplets multiplexed across S different samples, where their genotypes are available across V exonic variants. Let d_{cv} be the number of unique reads overlapping with the v-th variant from the c-th droplet. Let b_{cvi} ∈ {R, A, O}, i ∈ {1, . . . , d_{cv}} be the variant-overlapping base call from the i-th read, representing reference (R), alternate (A), and other (O) alleles respectively. Let e_{cvi} ∈ {0,1} be a latent variable indicating whether the base call is correct (0) or not (1), then given e_{cvi}=0, b_{cvi} ∈{R=0, A=1} and ˜Binomial

$$( 2 , \frac{g}{2} )$$

when g ∈ {0,1,2} is the true genotype of sample corresponding to c-th droplet at v-th variant. When e_{cvi}=1, it is assumed that Pr(b_{cvi}|g, e_{cvi}) follows a data table. e_{cvi} is assumed to follow Bernoulli

$$( {10}^{- \frac{{q}_{cvi}}{10}} )$$

where q_{cvi} is a q_{cvi} is a phred-scale quality score of the observed base call. In some embodiments, a workflow was used to process the raw reads, which estimates the phred-scale quality score based on the alignment of each read to the reference human transcriptome using an aligner.

In some embodiments, uncertainty of observed genotypes is allowed at the v-th variant for the s-th sample using Pₛᵥ^{(g)}=Pr(g|Datsₛᵥ), the posterior probability of a possible genotype g given external DNA data Dataₛᵥ (e.g. sequence reads, imputed genotypes, or array-based genotypes). If genotype likelihood Pr(Dataₛᵥ|g) is provided (e.g. unphased sequence reads) instead, it can be converted to a posterior probability scale using Pₛᵥ^{(g)}=Pr(Dataₛᵥ|g)Pr(g) where Pr(g)˜Binomial(2, pᵥ) and pᵥ is the population allele frequency of the alternate allele. To allow errors E in the posterior probability, it is possible to replace it to (1−ε)Pₛᵥ^{(g)}+εPr(g). Thus, the overall likelihood that the c-th droplet originated from the s-th sample is

L_{c}(s)=πᵥ₌₁^{V}[Σ_{g=0}²{πᵢ₌₁^{d}^{_{cv}})Σₑ₌₀¹Pr(b_{cvi}|g, e))Pₛᵥ^{(g)}]}

In certain embodiments having the absence of doublets, the maximum likelihood is used to determine the best-matching sample as argmaxₛ[L_{c}(s)]. Accordingly, in some embodiments, the second statistical comparison comprises: (i) determining a number of the sequence reads grouped into each of the partitions that overlap with the target variants of the unique variant combination associated with each subject, and (ii) determining a maximum likelihood that the sequence reads grouped into each of the partitions originated from each subject based on the determined number of the sequence reads grouped into each of the partitions that overlap with the target variants of the unique variant combination associated with each subject.

At step 650, a set of sequence data is compiled for each subject of the plurality of subjects. In some embodiments, the set of sequence data comprises the sequence reads grouped into each partition assigned to each subject of the plurality of subjects. In some embodiments, the sequence reads are organized by their partition-specific barcodes and the plurality of target variants into a matrix of sequence reads, partitions, and subjects for further analysis.

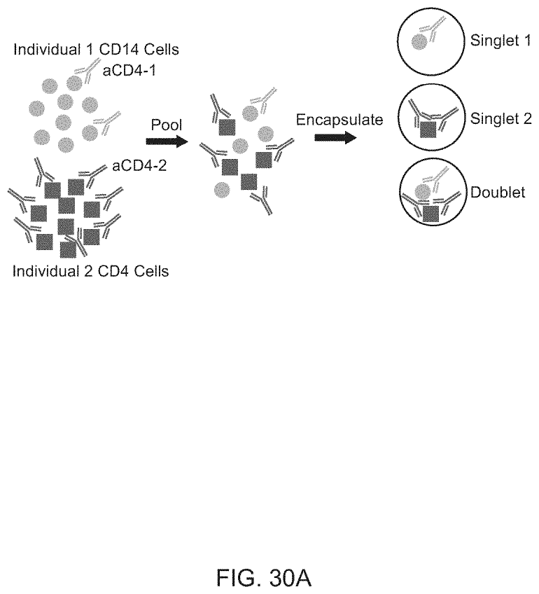

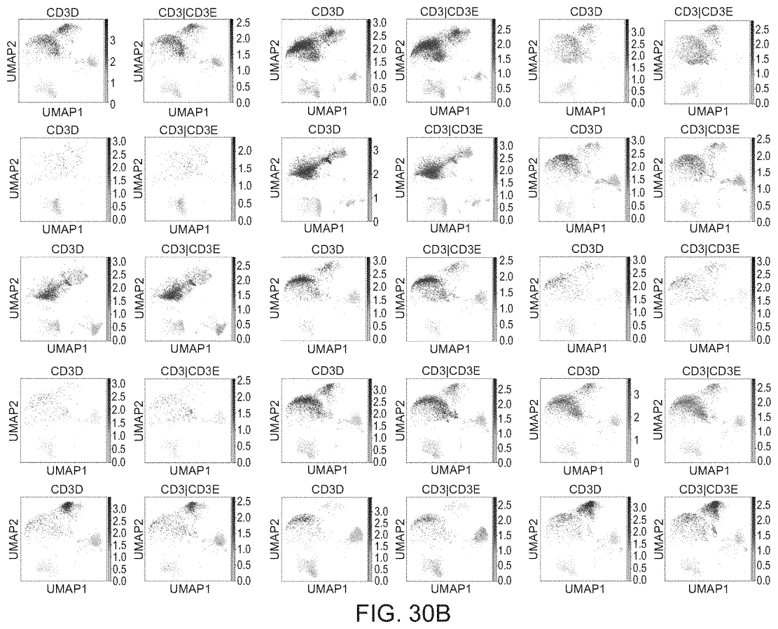

While multiplexed droplet-based sequencing of RNA (via cDNAs) using natural genetic barcodes are described herein, it should be noted the method can be adapted for other types of partition-based sequencing in which single cells or nuclei are introduced into separate partitions, assigned a partition-specific barcode, and grouped by subject according to natural genetic barcodes. As an example, for multiplexed scATAC-seq, demuxlet may utilize both coding and noncoding variants in chromatin accessible sites to assign the sequence reads grouped into each partition back to a reference genotyped subject. Alternatively, because scATAC-seq may be used to analyze the effect of a protein that cleaves or modifies genomic DNA where the cleavage is affected by chromatin conformation. Under different stimuli a piece of genomic DNA is more or less accessible to the cleaving protein. Thus, instead of measuring changes in RNA expression (e.g., coding and noncoding variants), demuxlet could be applied to measure changes in chromatin structure, for example, + or − stimulus. As another example, for multiplexed CITE-seq, the cell-specific barcode is captured in both the RNA transcripts as well as the amplified antibody-conjugated barcode library, thereby linking each cell's RNA transcripts with protein levels (see, e.g., FIG. 30A). In this instance, demuxlet may be applied to the single cell RNA-seq reads and then linked to the protein levels using the cell barcode.

### IV. Sequencing Samples and Analysis System

FIG. 7 shows an example expression profiling framework 700 used in accordance with various embodiments that includes one or more samples 705, such as molecular targets from a tissue or blood sample, or nucleic acid in a suspension, within a sample holder 710, e.g., a flow cell or a tube containing droplets of nucleic acid. A physical characteristic 715, such as a fluorescence intensity value, from the sample 705 is detected by a detector 720. A data signal 725 from the detector 720 can be sent to a data processing system 730 (onboard or separate from the detector), which may include a processor 750 and a memory 735. Data signal 725 may be stored locally in the data processing system 730 in memory 735, or externally in an external memory 740 or a storage device 745. Detector 720 can detect a variety of physical signals, such as light (e.g., fluorescent light from different probes for different bases) or electrical signals (e.g., as created from a molecule traveling through a nanopore). The data processing system 730 may be, or may include, a computer system, ASIC, microprocessor, etc., as described in further detail with respect to FIG. 8. The data processing system 730 may also include or be coupled with a display (e.g., monitor, LED display, etc.) and a user input device (e.g., mouse, keyboard, buttons, etc.). The data processing system 730 and the other components may be part of a stand-alone or network connected computer system, or they may be directly attached to or incorporated in a thermal cycler device. The data processing system 730 may also include optimization software that executes in processor 750. Based on the physical characteristic 715 obtained from the detector 720, sequencing data may be generated and variants in one or more reads may be identified, quantified and analyzed, by the data processing system, to determine a unique variant combination associated with each subject or sample.

In some embodiments, the expression profiling framework 700 further comprises a microfluidic device 755 for isolating and/or encapsulating target molecules in partitions together with partition-specific barcodes. The microfluidic device 755 may join two aqueous flows before their compartmentalization into discrete partitions. Laminar flow prevents mixing of the two aqueous inputs prior to compartmentalization into discrete partitions. One flow contains the target molecules such as cells, and the other flow contains partition-specific barcodes such as microparticles with partition-specific barcodes suspended in a lysis buffer. A data signal 760 from the microfluidic device 455 can be sent to the data processing system 730 (onboard or separate from the microfluidic device). Data signal 760 may be stored locally in the data processing system 730 in memory 735, or externally in an external memory 740 or the storage device 745. Microfluidic device 755 can detect a variety of physical signals, such as light (e.g., images from a microscope) or electrical signals (e.g., as created from flow sensors or feedback control loops).

Any of the computer systems or data processing systems described herein may utilize any suitable number of subsystems. An example of a computer system or data processing system (e.g., the data processing system 730 described with respect to FIG. 7) and associate subsystems is shown in FIG. 8. The computing system 800 is only one example of a suitable computing system and is not intended to suggest any limitation as to the scope of use or functionality of the present embodiments. Also, computing system 800 should not be interpreted as having any dependency or requirement relating to any one or combination of components illustrated in expression profiling framework 800.

As shown in FIG. 8, computing system 800 includes a computing device 505. The computing device 805 can be resident on a network infrastructure such as within a cloud environment, or may be a separate independent computing device (e.g., a computing device of a service provider). The computing device 805 may include a bus 810, processor 815, a storage device 820, a system memory (hardware device) 825, one or more input devices 830, one or more output devices 835, and a communication interface 840.

The bus 810 permits communication among the components of computing device 805. For example, bus 810 may be any of several types of bus structures including a memory bus or memory controller, a peripheral bus, and a local bus using any of a variety of bus architectures to provide one or more wired or wireless communication links or paths for transferring data and/or power to, from, or between various other components of computing device 805.

The processor 815 may be one or more conventional processors, microprocessors, or specialized dedicated processors that include processing circuitry operative to interpret and execute computer readable program instructions, such as program instructions for controlling the operation and performance of one or more of the various other components of computing device 805 for implementing the functionality, steps, and/or performance of the present invention. In certain embodiments, processor 815 interprets and executes the processes, steps, functions, and/or operations of the present invention, which may be operatively implemented by the computer readable program instructions. For example, processor 815 can retrieve, e.g., import and/or otherwise obtain or generate sequence data, query the sequence data, identify variants, deconvolute sequence reads, remove sequence reads from a matrix for partitions that are determined to be doublets, assign a subject from the plurality of subjects to partitions remaining in the matrix that have sequence reads, and perform statistical analysis, e.g., determining a number of the sequence reads grouped into each of the partitions that overlap with the target variants of the unique variant combination associated with each subject, and determining a maximum likelihood that the sequence reads grouped into each of the partitions originated from each subject based on the determined number of the sequence reads grouped into each of the partitions that overlap with the target variants of the unique variant combination associated with each subject. In some embodiments, the information obtained or generated by the processor 815, e.g., the sequence data or sequence reads, the identified variants, the partitions, statistical values, barcodes etc., can be stored in the storage device 820.

The storage device 820 may include removable/non-removable, volatile/non-volatile computer readable media, such as, but not limited to, non-transitory machine readable storage medium such as magnetic and/or optical recording media and their corresponding drives. The drives and their associated computer readable media provide for storage of computer readable program instructions, data structures, program modules and other data for operation of computing device 805 in accordance with the different aspects of the present invention. In embodiments, storage device 820 may store operating system 845, application programs 850, and program data 855 in accordance with aspects of the present invention.

The system memory 825 may include one or more storage mediums, including for example, non-transitory machine readable storage medium such as flash memory, permanent memory such as read-only memory (“ROM”), semi-permanent memory such as random access memory (“RAM”), any other suitable type of non-transitory storage component, or any combination thereof. In some embodiments, an input/output system 560 (BIOS) including the basic routines that help to transfer information between the various other components of computing device 805, such as during start-up, may be stored in the ROM. Additionally, data and/or program modules 865, such as at least a portion of operating system 845, program modules, application programs 850, and/or program data 855, that are accessible to and/or presently being operated on by processor 815, may be contained in the RAM. In embodiments, the program modules 865 and/or application programs 850 can comprise an index, matrix, or table of variants, barcodes, subjects, and partitions, algorithms or models such as a mixture model or maximum likelihood algorithm, a comparison tool, and a demuxlet tool which provides the instructions for execution of processor 815.

The one or more input devices 830 may include one or more mechanisms that permit an operator to input information to computing device 805, such as, but not limited to, a touch pad, dial, click wheel, scroll wheel, touch screen, one or more buttons (e.g., a keyboard), mouse, game controller, track ball, microphone, camera, proximity sensor, light detector, motion sensors, biometric sensor, and combinations thereof. The one or more output devices 835 may include one or more mechanisms that output information to an operator, such as, but not limited to, audio speakers, headphones, audio line-outs, visual displays, antennas, infrared ports, tactile feedback, printers, or combinations thereof.

The communication interface 840 may include any transceiver-like mechanism (e.g., a network interface, a network adapter, a modem, or combinations thereof) that enables computing device 805 to communicate with remote devices or systems, such as a mobile device or other computing devices such as, for example, a server in a networked environment, e.g., cloud environment. For example, computing device 805 may be connected to remote devices or systems via one or more local area networks (LAN) and/or one or more wide area networks (WAN) using communication interface 840.

As discussed herein, computing system 800 may be configured for multiplexed droplet-based sequencing using natural genetic barcodes. In particular, computing device 805 may perform tasks (e.g., process, steps, methods and/or functionality) in response to processor 815 executing program instructions contained in non-transitory machine readable storage medium, such as system memory 825. The program instructions may be read into system memory 825 from another computer readable medium (e.g., non-transitory machine readable storage medium), such as data storage device 820, or from another device via the communication interface 840 or server within or outside of a cloud environment. In embodiments, an operator may interact with computing device 805 via the one or more input devices 830 and/or the one or more output devices 835 to facilitate performance of the tasks and/or realize the end results of such tasks in accordance with aspects of the present invention. In additional or alternative embodiments, hardwired circuitry may be used in place of or in combination with the program instructions to implement the tasks, e.g., steps, methods and/or functionality, consistent with the different aspects of the present invention. Thus, the steps, methods and/or functionality disclosed herein can be implemented in any combination of hardware circuitry and software.

### V. Examples and Methods

The following examples and methods describe various use cases and analysis techniques that harnesses natural genetic variation to determine the sample identity of each partition containing a single cell (singlet) and detect partitions containing two cells (doublets). The specific use cases and analysis techniques are intended to be illustrative rather than limiting.

### EXAMPLE 1

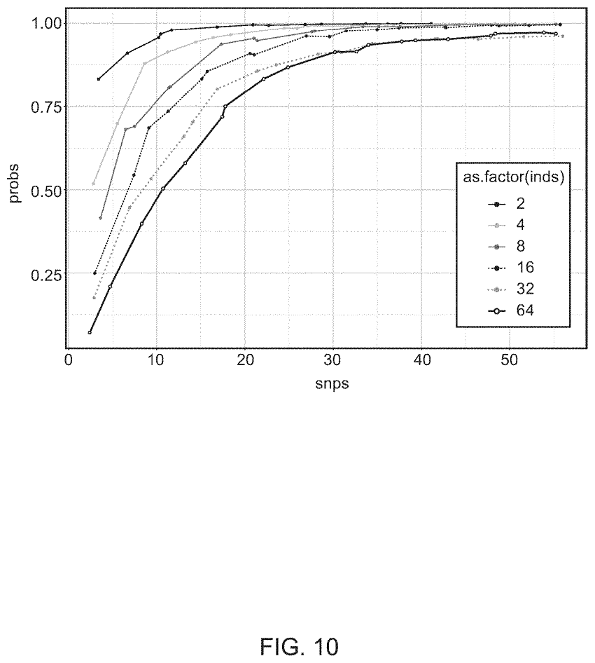

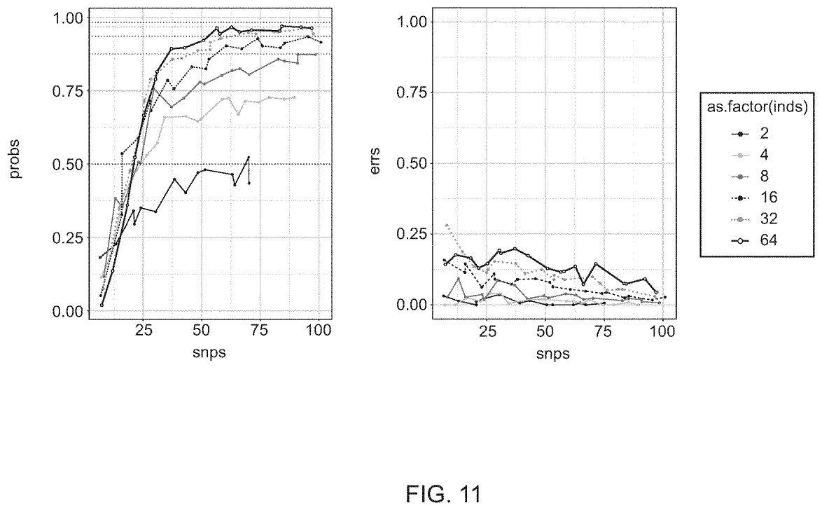

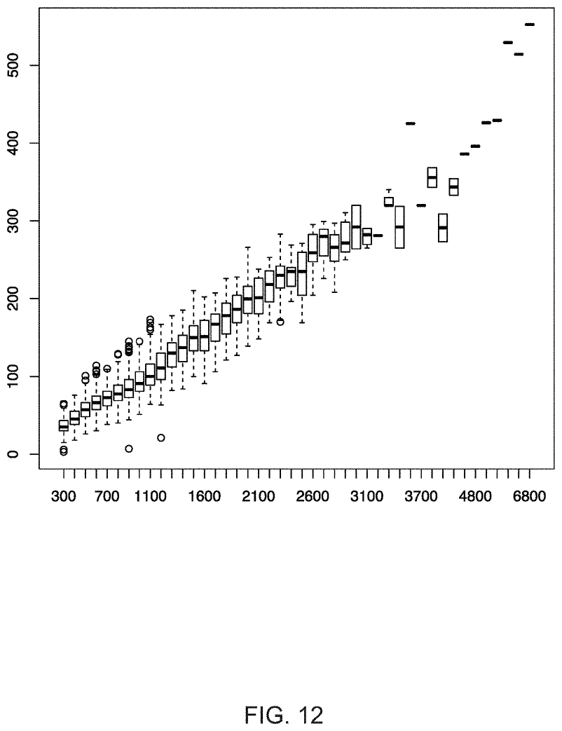

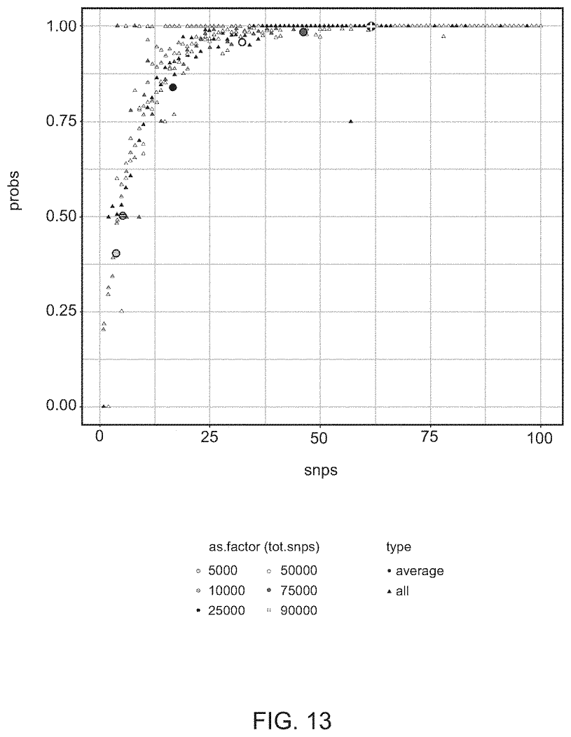

The performance of multiplexed droplet single-cell RNA-sequencing (dscRNA-seq) was assessed through simulation. The ability to demultiplex partitions such as droplets is a function of the number of individuals multiplexed, the depth of sequencing or number of read-overlapping SNPs, and the relatedness of multiplexed individuals. In this instance, 6,145 droplets (5,837 singlets and 308 doublets) were simulated from 2-64 individuals from the 1000 Genomes Project. It was demonstrated that 50 SNPs per droplet allows demultiplexing of 97% of singlets and identification of 92% of doublets in pools of up to 64 individuals (see, e.g., FIGS. 10 and 11). Simulating a range of sequencing depths, it was determined that 50 SNPs can be obtained with as few as 1,000 unique molecular identifiers (UMIs) per droplet (see, e.g., FIG. 12), and that the recommended sequencing depths of standard dscRNA-seq workflows would capture hundreds of SNPs. To assess dependence on the relatedness of multiplexed individuals, 6,145 singlets were simulated from a set of eight related individuals from 1000 Genomes. In this simulation, 50 SNPs per singlet would allow demuxlet to correctly assign over 98% of singlet (see, e.g., FIG. 13). These results suggest optimal multiplexed designs where cells from tens of unrelated individuals should be pooled, loaded at concentrations 2-10× higher than standard workflows, and sequenced to at least 1,000 UMIs per droplet.

### EXAMPLE 2

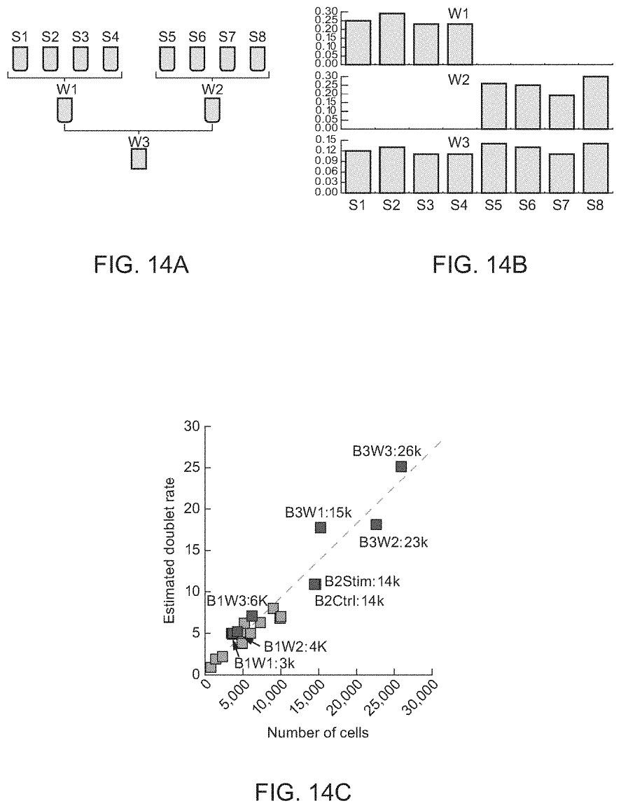

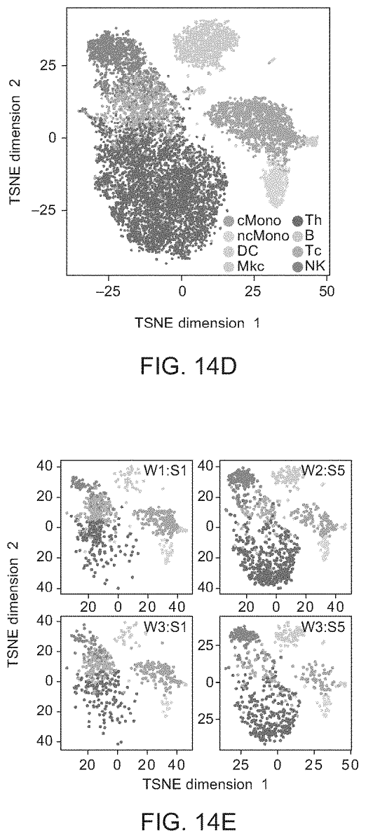

The performance of demuxlet was evaluated by analyzing a pool of peripheral blood mononuclear cells (PBMCs) from eight lupus patients. By sequential pairwise pooling, three pools of equimolar concentrations of cells were generated (W1: patients S1−S4, W2: patients S5−S8 and W3: patients S1−S8), and each were loaded in a well on a 10× Chromium instrument (see, e.g., FIG. 14A). 3,645 (W1), 4,254 (W2), and 6,205 (W3) cell-containing droplets were sequenced to an average depth of 51,000, 39,000, and 28,000 reads per droplet.

In wells W1, W2, and W3, demuxlet identified 91% (3,332/3,645), 91% (3,864/4,254), and 86% (5,348/6,205) of droplets as singlets (likelihood ratio test, L(singlet)/L(doublet)>2), of which 25% (±2.6%), 25% (±4.6%) and 12.5% (±1.4%) mapped to each donor, consistent with equal mixing of individuals in each well. From wells W1 and W2, each containing cells from two disjoint sets of four individuals, a demultiplexing error rate (number of cells assigned to individuals not in the pool) was estimated of less than 1% of singlets (W1: 2/3,332, W2: 0/3,864) (see, e.g., FIG. 14B).

### EXAMPLE 3

The performance of demuxlet was evaluated to detect doublets in both simulated and real data. 466/3,645 (13%) droplets from W1 were simulated as synthetic doublets by setting the cellular barcodes of 466 droplets, each from individuals S1 and S2, to be the same. Applied to simulated data, demuxlet identified 91% (426/466) of synthetic doublets as doublets or ambiguous, correctly recovering the sample identity of both cells in 403/426 (95%) doublets (see, e.g., FIG. 9). Applied to real data from W1, W2, and W3, demuxlet identified 138/3,645, 165/4,254, and 384/6,205 doublets corresponding to doublet rates of 5.0%, 5.2%, and 7.1%, consistent with the expected doublet rates estimated from mixed-species experiments (see, e.g., FIG. 14C).

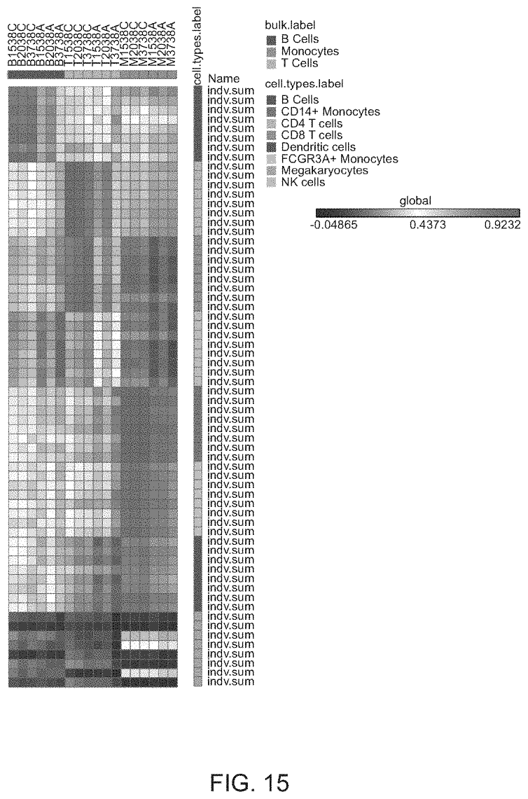

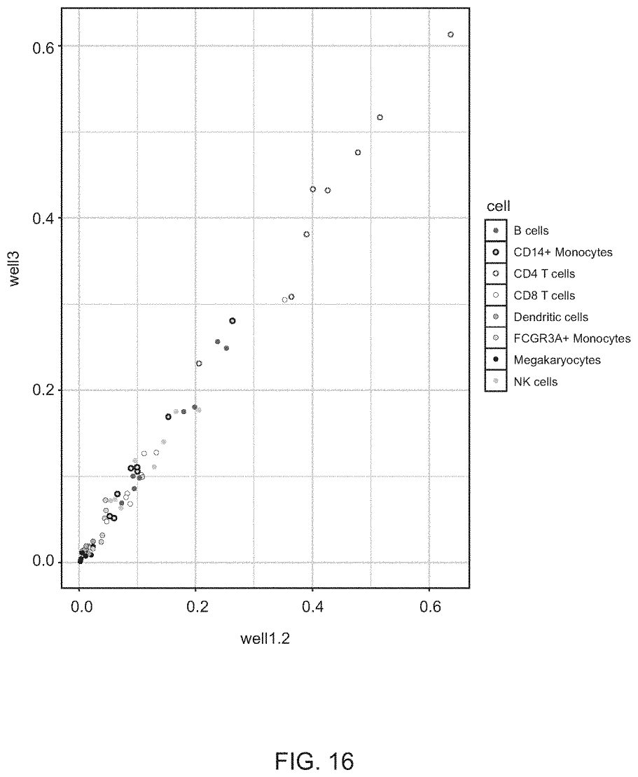

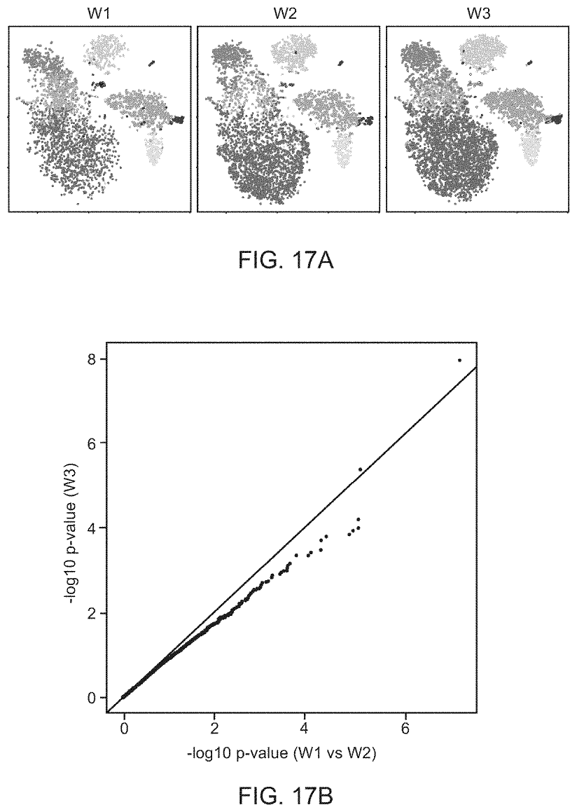

Demultiplexing of pooled samples allows for the statistical and visual comparisons of individual-specific dscRNA-seq profiles. Singlets identified by demuxlet in all three wells cluster into known immune cell types (see, e.g., FIG. 14D) and are correlated with bulk RNA-sequencing of sorted cell populations (R=0.76−0.92) (see, e.g., FIG. 15). For the same individuals from different wells, t-distributed stochastic neighbor embedding (t-SNE) of dscRNA-seq data are qualitatively consistent, and estimates of cell type proportions are highly correlated (R=0.99) (see, e.g., FIG. 14E and FIG. 16). Further, t-SNE projections of the pool and each individual are not confounded by well-to-well effects (see, e.g., FIG. 17A). While six genes were differentially expressed between wells W1 and W2 (DESeq2 on pseudobulk counts, FDR<0.05), only two genes were differentially expressed between W1 and W2 individuals in well W3 (FDR<0.05) (see, e.g., FIG. 17B), suggesting multiplexing reduces technical effects owing to separate sample processing.

### EXAMPLE 4

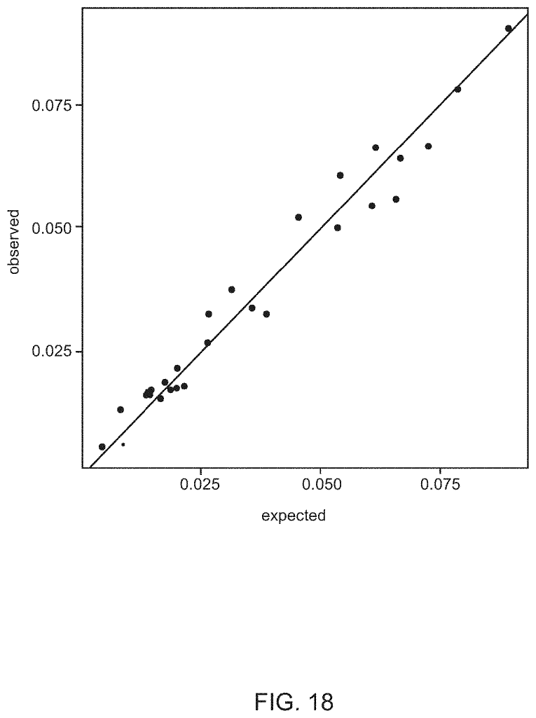

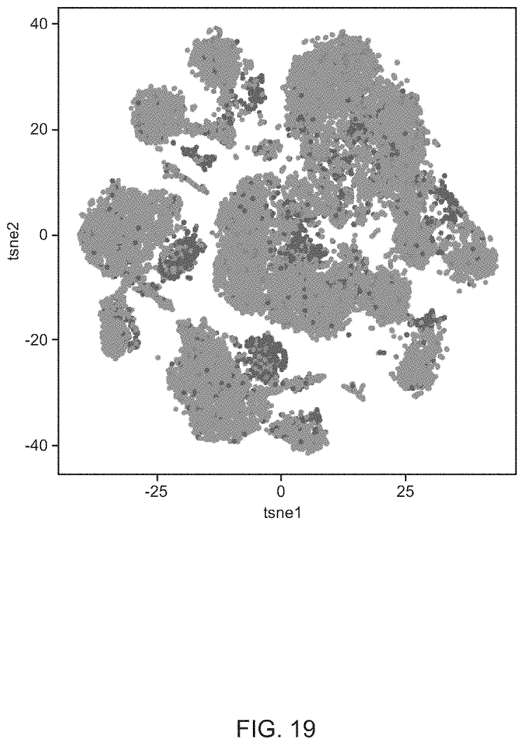

Multiplexed dscRNA-seq were used to characterize the cell-type specificity and inter-individual variability of response to IFN-β, a potent cytokine that induces genome-scale changes in the transcriptional profiles of immune cells14,15. From each of eight lupus patients, PBMCs were activated with recombinant IFN-β or left untreated for 6 h, a time point where it was previously found to maximize the expression of interferon-sensitive genes in dendritic cells and T cells. Two pools, IFN-β-treated and control, were prepared with the same number of cells from each individual and loaded onto the 10× Chromium instrument. In this instance 14,619 (control) and 14,446 (stimulated) cell-containing droplets were obtained, of which demuxlet identified 83% (12,138) and 84% (12,167), respectively, as singlets. The estimated doublet rate of 10.9% in each condition is consistent with predicted rates (see, e.g., FIG. 14C), and the observed and expected frequencies of doublets for each pair of individuals were highly correlated (R=0.98) (see, e.g., FIG. 18). Detected doublets form distinct clusters near the periphery of other clusters defined by cell type (see, e.g., FIG. 19).

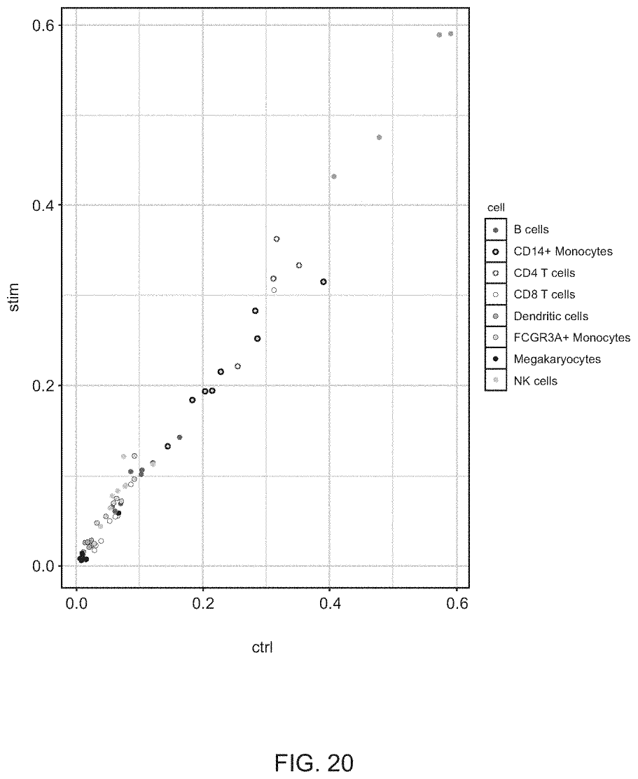

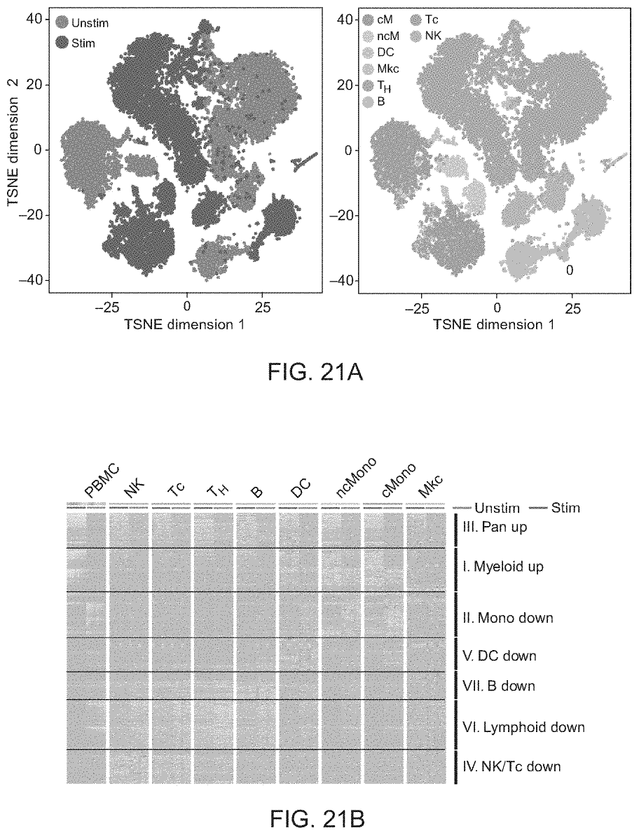

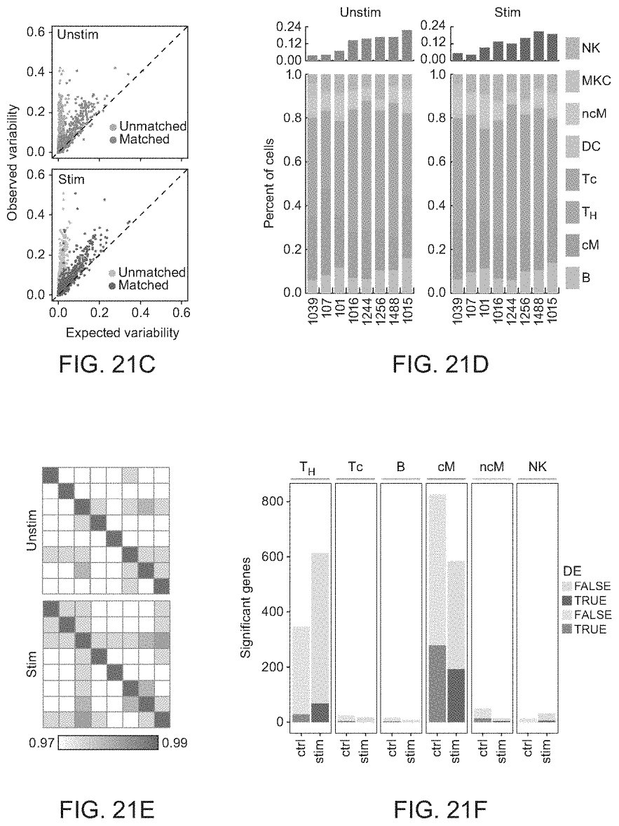

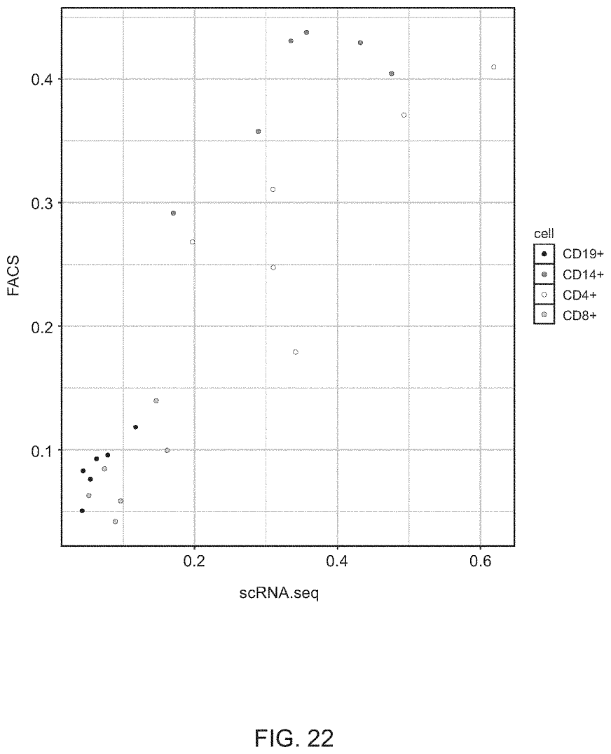

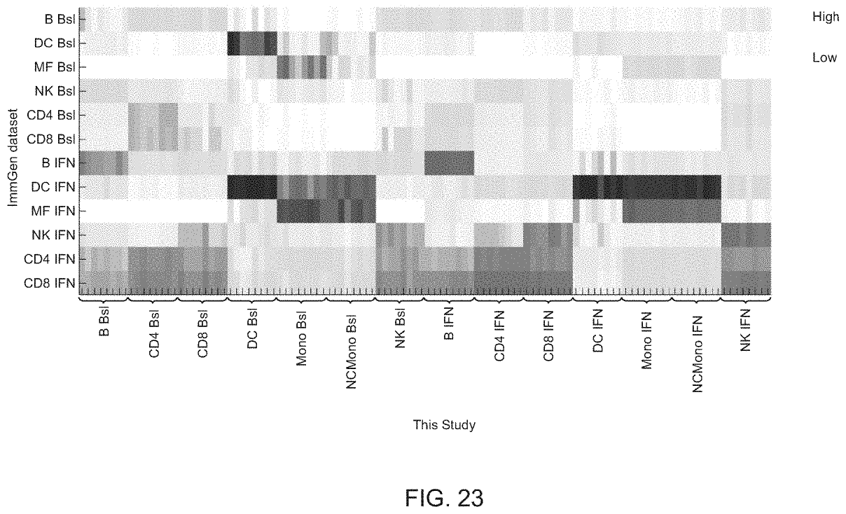

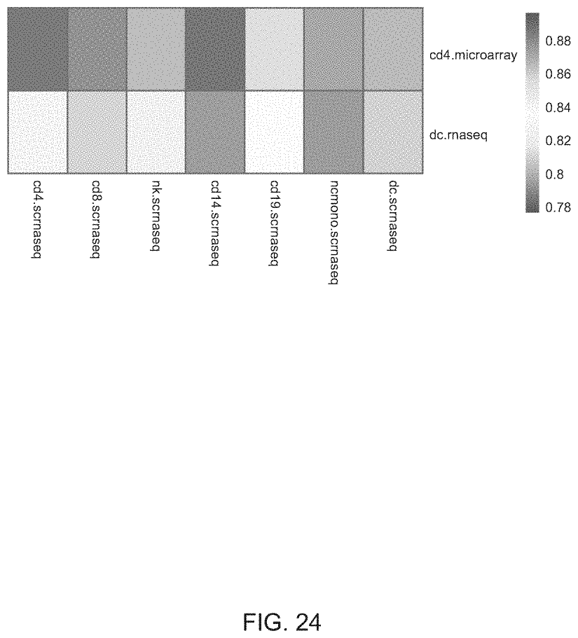

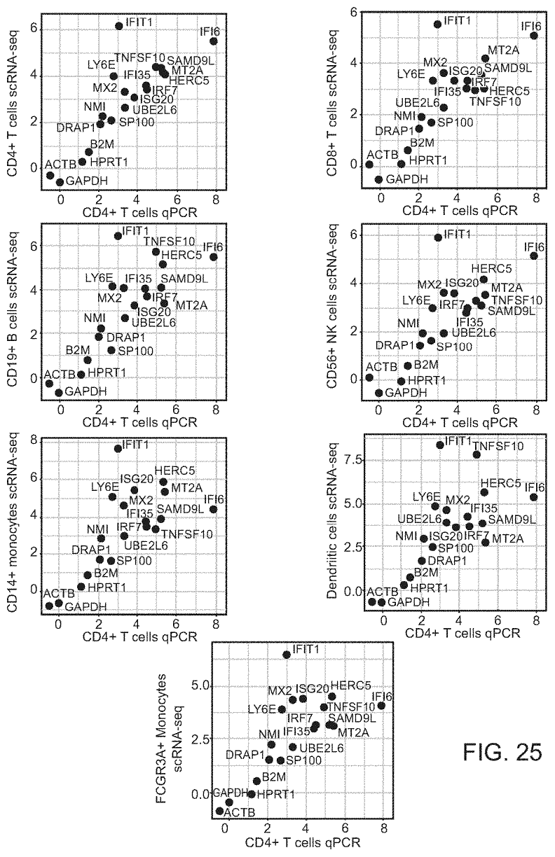

Demultiplexing individuals enabled the use of the eight individuals within each pool as biological replicates to quantitatively assess cell-type-specific IFN-β responses in PBMCs. Consistent with previous reports from bulk RNA-sequencing data, IFN-β stimulation induces widespread transcriptomic changes observed as a shift in the t-SNE projections of singlets (see, e.g., FIG. 20A). As expected, IFN-β did not affect cell type proportions between control and stimulated cells (see, e.g., FIG. 21), and these were consistent with flow cytometry measurements (R=0.88) (see, e.g., FIG. 22). Estimates of abundances for ˜2,000 homologous genes in each cell type and condition correlated with similar data from mice (see, e.g., FIG. 23). In this instance, 3,055 differentially expressed genes abs(logFC)>1 were identified, FDR<0.05) in at least one cell type. For 709 genes, estimates of fold change in response to IFN-β stimulation in myeloid and CD4+ cells were consistent with estimates in monocyte-derived dendritic cells and CD4+ T cells, respectively (see, e.g., FIG. 24) and correlated with qPCR results of sorted CD4+ T cells (see, e.g., FIG. 25). Differentially expressed genes clustered into modules of cell-type-specific responses enriched for distinct gene regulatory programs (see, e.g., FIG. 20B. For example, genes upregulated in all leukocytes (Cluster III 401 genes, abs(logFC)>1, FDR<0.05) or only in myeloid cells (Cluster I: 767 genes, abs(logFC)>1, FDR<0.05) were enriched for general antiviral response (e.g., KEGG Influenza A: Cluster III P<1.6×10−5), chemokine signaling (Cluster I P<7.6×10−3), and pathways active in systemic lupus erythematosus (Cluster I P<4.4×10−3). The five clusters of downregulated genes were enriched for antibacterial response (KEGG Legionellosis: Cluster II monocyte down P<5.5×10−3) and natural-killer-cell-mediated toxicity (Cluster IV NK/TH cell down: P<3.6×10−2). The analysis of multiplexed dscRNA-seq data recovers cell-type-specific gene regulatory programs affected by interferon stimulation consistent with published IFN-β signatures in mouse and humans.

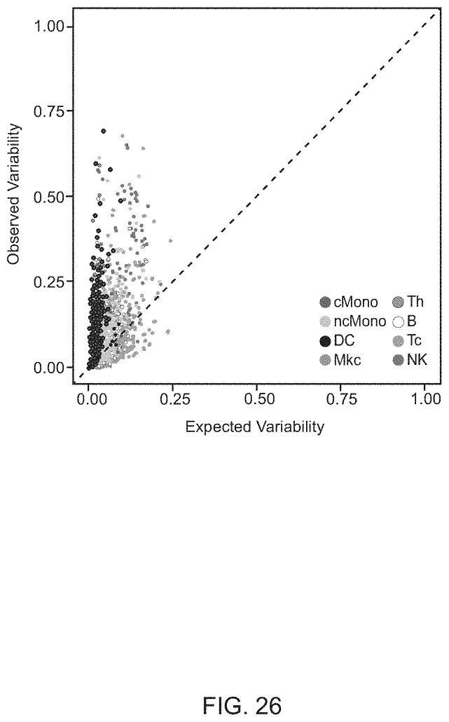

Over all PBMCs, the variance of mean expression across individuals was higher than the variance across synthetic replicates whose cells were randomly sampled (Lin's concordance=0.022, Pearson correlation=0.69, FIG. 20C). The variance across synthetic replicates whose cells were randomly sampled; matching for cell type proportions was more concordant with the variance across individuals (Lin's concordance=0.54, Pearson correlation=0.78, FIG. 20C and FIG. 20D), suggesting a contribution of cell type composition on expression variability. However, for each cell type, the variance across individuals was also higher than the variance across synthetic replicates (Lin's concordance=0.007−0.20), suggesting additional inter-individual variability not due to cell type composition (see, e.g., FIG. 26). In CD14+CD16− monocytes, the correlation of mean expression between pairs of synthetic replicates from the same individual (>99%) is greater than from different individuals (˜97%), further indicating inter-individual variation beyond sampling (see, e.g., FIG. 20E). In this instance 15 to 827 genes were found with statistically significant inter-individual variability in control cells and 7 to 613 were found in stimulated cells (Pearson correlation, FDR<0.05), with most found in classical monocytes (cM) and CD4+ helper T (TH) cells. Inter-individual variable genes in stimulated cM and to a lesser extent in TH cells (P<9.3×10−4 and 4.5×10−2, hypergeometric test, FIG. 20F) were enriched for differentially expressed genes, consistent with our previous discovery of more IFN-β response—expression quantitative trait loci (eQTLs) in monocyte-derived dendritic cells than in CD4+ T cells. Comparing these genes to 407 genes previously profiled in bulk monocyte-derived dendritic cells, it was found that the proportion of variance explained by inter-individual variability was correlated more strongly in myeloid cells after stimulation (R =0.26−0.3) than before (R=0.05−0.19).

### EXAMPLE 5

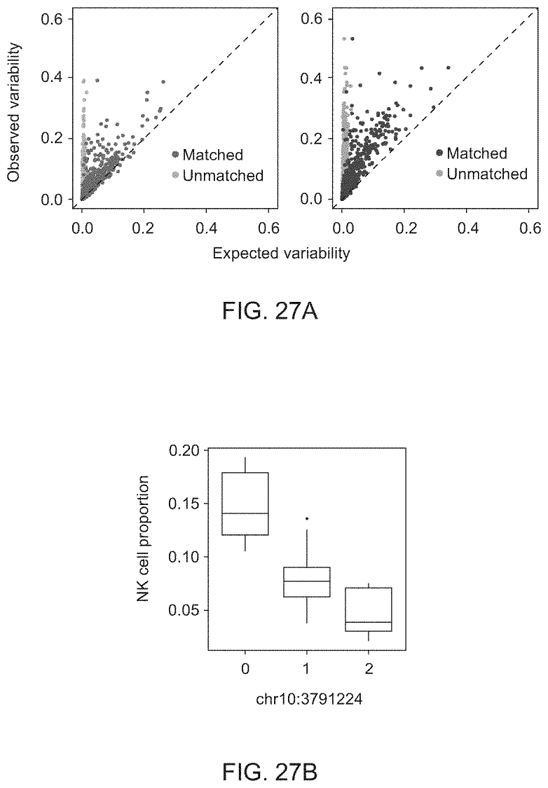

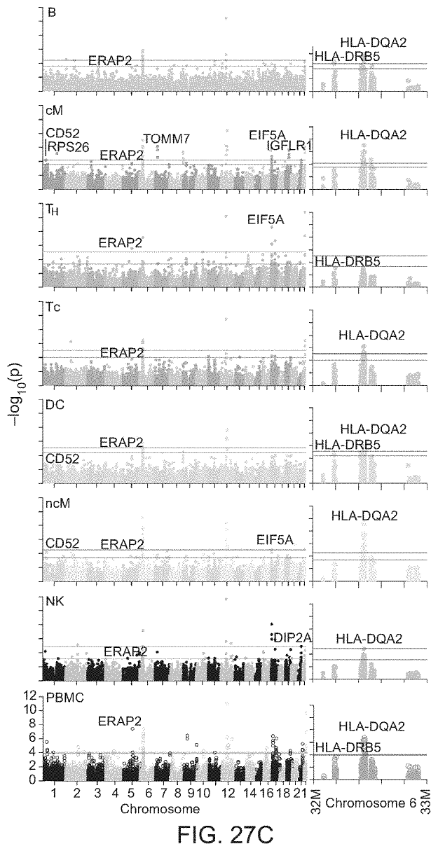

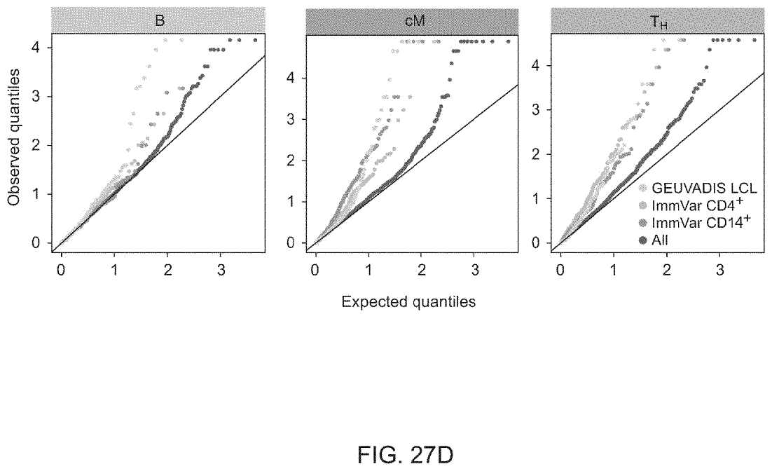

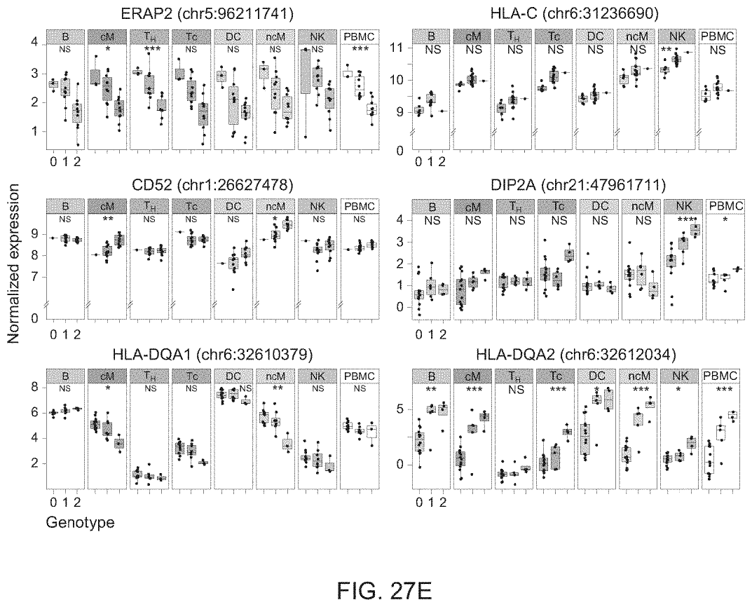

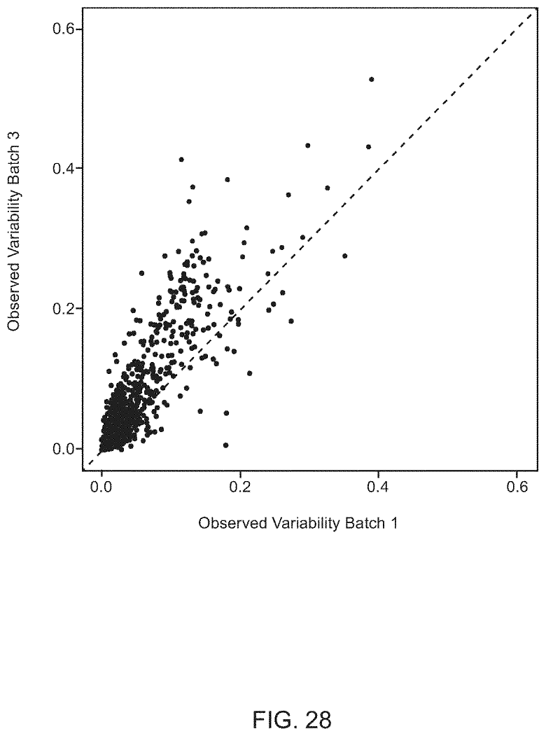

To map genetic variants associated with cell type proportions and cell-type-specific expression using multiplexed dscRNA-seq, an additional 15,250 (7 donors), 22,619 (8 donors), and 25,918 droplets (15 donors; 8 lupus patients, 5 rheumatoid arthritis patients, and 2 healthy controls) were sequenced. Demuxlet identified 71% (10,766/15,250), 73% (16,618/22,619), and 60% (15,596/25,918) of droplets, respectively, as singlets, correctly assigning 99% of singlets from the first two pools, W1 (10,740/10,766) and W2 (16,616/16,618). The estimated doublet rates of 18%, 18%, and 25% are consistent with the increased concentrations of loaded cells (FIG. 14C). Similar to the IFN-β stimulation experiment, it was found that expression variability was determined by variability in cell type proportion (see, e.g., FIG. 27A) and reproducible between batches (see, e.g., FIG. 28). Associating>150,000 genetic variants (MAF>20%) with the proportion of eight major Immune cell populations, a SNP (chr10:3791224) was identified significantly associated (P=1.03×10−5, FDR<0.05) with the proportion of natural killer (NK) cells (see, e.g., FIG. 27B).

### EXAMPLE 6

Across 23 donors, an eQTL analysis was conducted to map genetic variants associated with expression variability in each major immune cell type. A total of 32 local eQTLs (±100 kb, FDR<0.1) were found, 22 of which were detected in only one cell type (see, e.g., FIG. 25C). Previously reported local eQTLs from bulk CD14+ monocytes, CD4+ T cells, and lymphoblastoid cell lines were more significantly associated with gene expression in the most similar cell types (cM, TH, and B cells, respectively) than other cell types (FIG. 25D). An inverse variance weighted meta-analysis was used to identify genes with pan-cell-type eQTLs, including those in the major histocompatibility complex (MHC) class I antigen presentation pathway, such as, ERAP2 (P<3.57×10-32, meta-analysis), which encodes an aminopeptidase known to cleave viral peptides20, and HLA-C (P<1.74×10−29, meta-analysis), which encodes the MHC class I heavy chain (FIG. 25E). HLA-DQA1 has local eQTLs only in some cell types (P<2.11×10-15, Cochran's Q) while HLA-DQA2 has local eQTLs in all antigen presentation cells (P<1.02×10-43, Cochran's Q). Among other cell-type-specific local eQTLs are CD52, a gene ubiquitously expressed in leukocytes, which only has eQTLs in monocyte populations, and DIP2A, a gene with an eQTL only in NK cells, which is associated with immune response to vaccination in peripheral blood. These results demonstrate the ability of multiplexed dscRNA-seq to characterize inter-individual variation in immune response and when integrated with genetic data, reveal cell-type-specific genetic control of gene expression, which would be undetectable when bulk tissues are analyzed.

### EXAMPLE 7

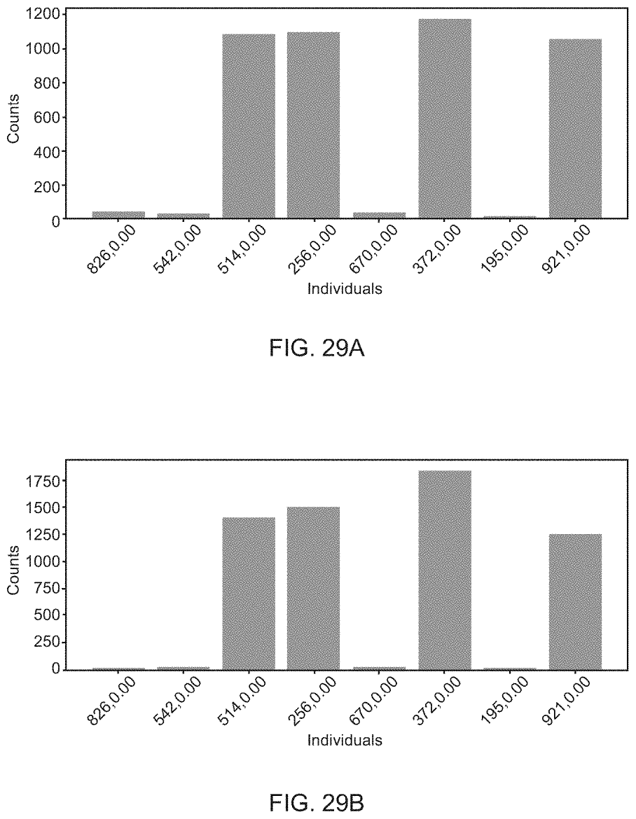

PBMCs from 4 previously-genotyped donors were isolated and cultured in 3 types of cell culture media: (1) supplemented with interferon gamma, (2) supplemented with interferon beta, and (3) unsupplemented. After culturing for 6 hours, cells were pooled, washed, and lysed to isolate their nuclei. Nuclei underwent transposition using hyperactive Tn5 transposase and then loaded onto two lanes of the 10× Chromium instrument, one with a low nuclei concentration (FIG. 29A) and one with a higher concentration (FIG. 29B). After aligning genomic reads from each droplet, demuxlet and known genotype information from these individuals were used to determine which droplets contained nuclei belonging to the same individual, and which droplets contained nuclei from two individuals. To test the accuracy of demuxlet, the genotypes of the 4 individuals in the study as well as those of 4 other individuals (whose cells were not included) were added as input to the method to examine any false positive demuxlet calls. In this case, demuxlet assigned less than 1% of cells to the 4 individuals that were included in the experiment.

### EXAMPLE 8

Peripheral blood cells from 15 individuals were thawed, isolated, and pooled in equal numbers per individual. The 99x BD Abseq panel was then used to stain the pooled cells for 45 min. After staining, cell suspensions were prepared and loaded onto the 10× Genomics Chromium Controller and processed with the 3′ V3 Kit. Library prep and sequencing were performed according to the standard protocol, and Abseq barcodes were amplified in a separate reaction and sequenced. The resulting genomic reads were used to match each cell to an individual based on reference genotypes for the 15 individuals and were correlated with protein levels from each cell. As an example, single cell gene expression of CD3D was highly concordant with protein levels across all individuals (FIG. 30B).

### Conclusion

The capability to demultiplex and identify doublets using natural genetic variation reduces the per-sample and per-cell library preparation cost of single-cell RNA-seq, ATAC-seq, and CITE-seq, removes the need for synthetic barcodes or split-pool strategies, and allows the capture of biological variability among individual samples while limiting unwanted technical variability. In various embodiments, it was found that an preferred number of samples to multiplex is ˜20, based on sample processing time and empirical doublet rates of current microfluidic devices, and anticipate that number to increase with automated sample handling and lower doublet rates.

Compared to sorting known cell types followed by bulk RNA-seq, ATAC-seq, and CITE-seq, multiplexed dscRNA-seq, dscATAC-seq, and dscCITE-seq are more efficient and unbiased methods for obtaining cell-type-specific immune traits. Demuxlet enables reliable estimation of cell type proportion, recovers cell-type specific transcriptional response to stimulation, and could facilitate further genetic and longitudinal analyses in relevant cell types and conditions across a range of sampled individuals, including between healthy controls and patients.

### Methods

Theoretical expectation of deconvoluting singlets.

The theoretical distribution of expected singlets with multiplexing (see, e.g., FIG. 27) is as follows. Let dₒ (e.g. 0.01) be the proportion of true multiplets when xₒ (1,000) cells are loaded when multiplexing was not used. Then the expected multiplet rates when x cells

are loaded can be modeled exponentially as

$$d ( x ) = 1 - {( 1 - {d}_{0} )}^{- \frac{x}{{x}_{0}}} .$$

Let α be the fraction of true singlets incorrectly classified as non-singlets (i.e. doublet or ambiguous), and β be the fraction of multiplets correctly classified as non-singlets. When multiplexing x cells equally

$$\frac{1}{n} d ( x )$$

from n samples, the expected multiplet rates are d(x) and are expected to be undetectable doublets mixed between the cells from the same sample. Therefore, the overall effective multiplet rate is

$$[ \frac{n - ( n - 1 ) β}{n} ] d ( x ) .$$

Similarly, the expected number of correctly identified singlets becomes

$$\frac{{x}_{0} ( 1 - α ) d ( x ) [ 1 - d ( x ) ]}{- log ( 1 - {d}_{0} )} .$$

Given α, β is me expected number of singlet can be calculated by fixing the multiplet rate d(x)=d₀. d₀=0.01, x₀=1000 was used for the simulation in FIG. 27.

### Dependence of Demultiplexing Performance on Experimental Design Parameters

The demuxlet ‘plp’ option was used to generate a pileup format of 6145 cells from one well of PBMC 10x data. The reads in the pileup were then modified to reflect the genotypes of individuals sampled from 1 kg. The pileup was downsampled to obtain different numbers of read-overlapping exonic SNPs (ranging from 5,000 to 100,000) for the whole cohort. To create simulated doublets, barcodes were randomly sampled and merged into pairs of barcodes within a data set, resulting in a 5% doublet rate in the original data. For simulations with related individuals, transcriptomes were simulated from eight individuals in 1000 Genomes with varying degrees of relatedness, ranging from unrelated to parent—child (HG00146, HG00147, HG00500, HG00501, HG00502, HG00512, HG00514, and HG00524).

### Isolation and Preparation of PBMC Samples.

Peripheral blood mononuclear cells were isolated from patient donors, Ficoll separated, and cryopreserved by the UCSF Core Immunologic Laboratory (CIL). PBMCs were thawed in a 37° C. water bath, and subsequently washed and resuspended in EasySep buffer (STEMCELL Technologies). Cells were treated with DNasel and incubated for 15 min at RT before filtering through a 40-μm column. Finally, the cells were washed in EasySep and resuspended in lx PBMS and 0.04% bovine serum albumin. Cells from eight donors were then re-concentrated to 1 M cells per mL and then serially pooled. At each pooling stage, 1 M cells per mL were combined to result in a final sample pool with cells from all donors.

### IFN-β Stimulation and Culture.

Prior to pooling, samples from eight individuals were separated into two aliquots each. One aliquot of PBMCs was activated by 100 U/mL of recombinant IFN-β (PBL Assay Science) for 6 hrs according to the published protocol. The second aliquot was left untreated. After 6 hrs, the 8 samples for each condition were pooled together in two final pools (stimulated cells and control cells) as described above.

### Fluorescence-Activated Cell Sorting and Analysis.

1M PBMCs from each donor were stained using standard procedure (30 min, 4 C) with the following surface antibody panel (CD3-PerCP (BD), CD4-APC (BD), CD8-BV570 (BioLegend), CD14-FITC (BD), CD19-BV510 (BioLegend, CD56-PECy7 (BD), and Ghost dye A710 viability stain (Tonbo)). Samples were then analyzed and sorted using a BD FACSAria Fusion instrument at the UCSF flow cytometry core. To calculate cell type proportions, the number of events in each of CD3+ CD4+ CD8⁻(CD4+ T cells), CD3+ CD4⁻ CD8+ (CD8+ T cells), CD3⁻ CD19+ (B cells), CD3⁻ CD14+ (monocytes), and CD3⁻ CD56⁺ gates (NK cells) was divided by the sum of events in these gates (see, e.g., FIG. 28).

### Quantitative Polymerase Chain Reaction Analysis.

RNA was isolated from sorted CD4+ T cells following the RNeasy micro kit protocol (QIAGEN), and cDNA was prepared using MultiScribe Reverse Transcriptase (Applied Biosystems cat #4368814). The qPCR primers were chosen from the PrimerBank reference when available32. Each sample was run in triplicate with the Luminaris HiGreen qPCR kit (Thermo Scientific #K0992) according to standard protocol using a Roche Light Cycler 96 instrument and fold change was calculated from AACT between control and stimulated samples with GAPDH as a reference gene.

### Droplet-Based Capture and Sequencing.

Cellular suspensions were loaded onto the 10× Chromium instrument (10× Genomics) and sequenced as described in Zheng, G. X. et al. Massively parallel digital transcriptional profiling of single cells. Nat. Commun. 8, 14049 (2017). The cDNA libraries were sequenced using a custom program on ten lanes of Illumina HiSeq2500 Rapid Mode, yielding 1.8 B total reads and 25 K reads per droplet. At these depths, >90% of captured transcripts were recovered in each sequencing experiment.

Bulk Isolation and Sequencing.

PBMCs from lupus patients were isolated and prepared as described above. Once resuspended in EasySep buffer, the EasyEights Magnet was used to sequentially isolate CD14+ (using the EasySep Human CD14 positive selection kit II, cat #17858), CD19+ (using the EasySep Human CD19 positive selection kit II, cat #17854), CD8+ (EasySep Human CD8 positive selection kitII, cat#17853), and CD4+ cells (EasySep Human CD4 T cell negative isolation kit (cat #17952) according to the kit protocol. RNA was extracted using the RNeasy Mini kit (#74104), and reverse transcription and tagmentation were conducted according to Picelli et al. using the SmartSeq2 protocol. After cDNA synthesis and tagmentation, the library was amplified with the Nextera XT DNA Sample Preparation Kit (#FC-131-1096) according to protocol, starting with 0.2 ng of cDNA. Samples were then sequenced on one lane of the Illumina Hiseq4000 with paired end 100-bp read length, yielding 350 M total reads.

### Alignment and Initial Processing of Single Cell Sequencing Data.

A CellRanger with v1.1 and v1.2 software was used with the default settings to process the raw FASTQ files, align the sequencing reads to the hg19 transcriptome, and generate a filtered UMI expression profile for each cell. The raw UMI counts from all cells and genes with nonzero counts across the population of cells were used to generate t-SNE profiles.

### Cell Type Classification and Clustering.

To identify known immune cell populations in PBMCs, the Seurat package was used to perform unbiased clustering on the 2.7 k PBMCs from Zheng et al., following the publicly available Guided Clustering Tutorial. The FindAllMarkers function was then used to find the top 20 markers for each of the eight identified cell types. Cluster averages were calculated by taking the average raw count across all cells of each cell type. For each cell, the Spearman correlation of the raw counts of the marker genes and the cluster averages were calculated, and assigned each cell to the cell type to which it had maximum correlation.

### Differential Expression Analysis.

Demultiplexed individuals were used as replicates for differential expression analysis. For each gene, raw counts were summed for each individual. The DESeq2 package was used to detect differentially expressed genes between control and stimulated conditions. Genes with baseMean>1 were filtered out from the DESeq2 output, and the qvalue package was used to calculate FDR<0.05.

### Estimation of Interindividual Variability in PBMCs.

For each individual, the mean expression was found of each gene with nonzero counts. The mean was calculated from the log2 single-cell UMI counts normalized to the median count for each cell. To measure inter-individual variability, the variance of the mean expression was calculated across all individuals. Lin's concordance correlation coefficient was used to compare the agreement of observed data and synthetic replicates. Synthetic replicates were generated by sampling without replacement either from all cells or cells matched for cell type proportion. Cell-type-specific variability estimated as the correlation between synthetic replicates was compared to variability estimates from 23 biological replicates of bulk IFN-stimulated monocyte-derived dendritic cells. Protein coding genes (407/414) originally measured using Nanostring (a hybridization based PCRfree quantification method) were assessed, and variability in the bulk data set was estimated as repeatability using a linear mixed model.

### Estimation of Interindividual Variability Within Cell Types.

For each cell type, two bulk equivalent replicates were generated for each individual by summing raw counts of singlets sampled without replacement. DESeq2 was used to generate variance-stabilized counts across all replicates. To filter for expressed genes, all subsequent analyses were performed on genes with 5% of samples with >0 counts. The correlation of replicates was performed on the log2-normalized counts. Pearson correlation of the two replicates from each of the eight individuals was used to find genes with significant inter individual variability.

### Quantitative Trait Mapping in Major Immune Cell Types.

Genotypes were imputed with EAGLE and filtered for MAF>0.2, resulting in a total of 189,322 SNPs. Cell type proportions were calculated as number of cells for each cell type divided by the number of total cells for each person. Linear regression was used to test associations between each genetic variant and cell-type proportion with the Matrix eQTL software40. Cis-eQTL mapping was conducted in each cell type separately. All genes with at least 50 UMI counts in 20% of the individuals in all PBMCs were tested for each cell type, resulting in a total of 4,555 genes. Variance-stabilized and log-normalized gene expression was calculated using the ‘dog’ function of the DESeq2 package36. All variants within a window of 100 kbp of each gene were tested with linear regression using Matrix eQTL40. Batch information for each sample as well as the first three principal components of the expression matrix were used as covariates.

While the invention has been described in detail, modifications within the spirit and scope of the invention will be readily apparent to the skilled artisan. It should be understood that aspects of the invention and portions of various embodiments and various features recited above and/or in the appended claims may be combined or interchanged either in whole or in part. In the foregoing descriptions of the various embodiments, those embodiments which refer to another embodiment may be appropriately combined with other embodiments as will be appreciated by the skilled artisan. Furthermore, the skilled artisan will appreciate that the foregoing description is by way of example only, and is not intended to limit the invention.

1-34. (canceled)

35. A method comprising:
obtaining, by a data processing system, genotypes of multiple different loci for a plurality of subjects, wherein the different loci comprise a predetermined set of target variants that provide a unique variant combination associated with each subject from the plurality of subjects;
obtaining, by the data processing system, sequence reads for nucleic acid from target molecules of the plurality of subjects, wherein the sequence reads comprise nucleic acid sequences with associated partition-specific barcodes that identify a partition from which each sequence read originated;
deconvoluting, by the data processing system, the sequence reads by the partition-specific barcodes to group the sequence reads by the partition from which each sequence read originated;
identifying, by the data processing system, a plurality of target variants in the sequence reads grouped into each of the partitions; and
assigning, by the data processing system, at least one subject from the plurality of subjects to one or more partitions based on a statistical comparison of the unique variant combination associated with the each subject from the plurality of subjects and the plurality of target variants in the sequence reads grouped into the one or more partitions.

36. The method of claim 35, wherein the statistical comparison comprises: (i) determining a number of the sequence reads grouped into each of the partitions that overlap with the target variants of the unique variant combination associated with each subject, and (ii) determining a maximum likelihood that the sequence reads grouped into each of the partitions originated from each subject based on the determined number of the sequence reads grouped into each of the partitions that overlap with the target variants of the unique variant combination associated with each subject.

37. The method of claim 36, wherein the variants from the set of target variants are single nucleotide polymorphisms (SNPs).

38. The method of claim 37, wherein the deconvoluting comprises organizing the sequence reads by the partition-specific barcodes into a matrix of sequence reads and partitions.

39. The method of claim 38, wherein prior to the assigning:
determining, by the data processing system, whether the sequence reads grouped into each of the partitions originate from more than one subject from the plurality of subjects;
when the sequence reads grouped into a partition do originate from more than one subject from the plurality of subjects, the sequence reads associated with the partition are removed from the matrix; and
when the sequence reads grouped into a partition do not originate from more than one subject from the plurality of subjects, performing the assigning of a subject to the partition based on the statistical comparison of the unique variant combination associated with the each subject from the plurality of subjects and the plurality of target variants in the sequence reads grouped into the partition.

40. The method of claim 39, wherein the determining whether the sequence reads grouped into each of the partitions originate from more than one subject from the plurality of subjects comprises generating a mixture model to calculate a likelihood that the sequence reads grouped into each partition originates from more than one subject from the plurality of subjects.

41. The method of claim 40, further comprising compiling, by the data processing system, a set of sequence data for each subject of the plurality of subjects, wherein the set of sequence data comprises the sequence reads grouped into each partition assigned to each subject of the plurality of subjects, and the compiling comprises organizing the sequence reads by the partition-specific barcodes and the plurality of target variants into a matrix of sequence reads, partitions, and subjects for further analysis.

42. A method comprising:
obtaining, by a data processing system, genotypes of multiple different loci for a plurality of subjects, wherein the different loci comprise a predetermined set of target variants that provide a unique variant combination associated with each subject from the plurality of subjects;
obtaining, by the data processing system, sequence reads for nucleic acid from the plurality of subjects, wherein the sequence reads comprise nucleic acid sequences with associated partition-specific barcodes that identify a partition from which each sequence read originated;
deconvoluting, by the data processing system, the sequence reads by the partition-specific barcodes to group the sequence reads by the partition from which each sequence read originated wherein the deconvoluting comprises organizing the sequence reads by the partition-specific barcodes into a matrix of sequence reads and partitions;
determining, by the data processing system, a presence of a plurality of target variants in the sequence reads grouped into each of the partitions, wherein the plurality of target variants determined in the sequence reads grouped into each of the partitions are added to the matrix of sequence reads and partitions;
removing sequence reads from the matrix for partitions that are determined to be doublets based on a first statistical comparison of the unique variant combination associated with each subject and the plurality of target variants in the sequence reads grouped into each of the partitions; and
assigning a subject from the plurality of subjects to the partitions remaining in the matrix that have sequence reads based on a second statistical comparison of the unique variant combination associated with the each subject from the plurality of subjects and the plurality of target variants in the sequence reads grouped into the remaining partitions.

43. The method of claim 42, wherein the first statistical comparison comprises generating a mixture model to calculate a likelihood that the sequence reads grouped into each partition originates from more than one subject from the plurality of subjects.

44. The method of claim 43, wherein the second statistical comparison comprises: (i) determining a number of the sequence reads grouped into each of the partitions that overlap with the target variants of the unique variant combination associated with each subject, and (ii) determining a maximum likelihood that the sequence reads grouped into each of the partitions originated from each subject based on the determined number of the sequence reads grouped into each of the partitions that overlap with the target variants of the unique variant combination associated with each subject.

45. The method of claim 44, wherein the variants from the set of target variants are single nucleotide polymorphisms (SNPs) and the set comprises at least fifty SNPs.

46. The method of claim 44, further comprising compiling, by the data processing system, a set of sequence data for each subject of the plurality of subjects, wherein the set of sequence data comprises the sequence reads grouped into each partition assigned to each subject of the plurality of subjects, and the compiling comprises organizing the sequence reads by the partition-specific barcodes and the plurality of target variants into a matrix of sequence reads, partitions, and subjects for further analysis.

47. A method for multiplex analysis of samples from different individuals, the method comprising:
pooling a plurality of samples from different individuals to produce a pooled sample;
conducting a sequencing reaction on the pooled sample to produce a plurality of sequence reads; and
associating each of the plurality of sequence reads back to the different individuals by identifying unique variants in each of the plurality of sequence reads and correlating the unique variants in each of the plurality of sequence reads to previously obtained sequence information of each different individual, wherein a highest match of the unique variants in each of the plurality of sequence reads to an individual's previously obtained sequence information determines which sequence read is associated with which different individual.

48. The method of claim 47, wherein conducting a sequencing reaction comprises:
generating a plurality of partitions, each partition comprising a single cell from a different individual; and
sequencing nucleic acid from each single cell from each of the plurality of partitions to generate a plurality of sequence reads, wherein each of the plurality of sequence reads comprises a partition specific barcode.

49. The method of claim 48, wherein the method further comprises: associating each of the plurality of sequence reads back to one of the plurality of partitions based on the partition specific barcode.

50. The method of claim 47, wherein the single cell is an immune cell.

51. The method of claim 49, wherein duplicate sequence reads of the plurality of sequence reads are removed prior to the associating steps.

52. The method of claim 47, wherein the correlating step further comprises: performing a statistical comparison of the unique variants with each different individual's previously obtained sequence information.

53. The method of claim 52, wherein the statistical comparison comprises: (i) determining a number of the plurality of sequence reads grouped into each of the partitions that overlap with each different individual's previously obtained sequence information, and (ii) determining a maximum likelihood that the plurality of sequence reads grouped into each of the partitions originated from an individual based on the determined number of the plurality of sequence reads grouped into each of the partitions that overlap with each different individual's previously obtained sequence information.

54. The method of claim 49, wherein the first associating step comprises organizing the plurality of sequence reads by the partition-specific barcodes into a matrix of sequence reads and partitions.
